(19) United States
(12) Patent Application Publication
(10) Pub. No.: US 2026/0235626 A1
(43) Pub. Date: Aug. 13, 2026

(54) BIOMARKERS FOR HUNTINGTON DISEASE STRATIFICATION, METHODS AND USES THEREOF

(71) Applicant: THE UNIVERSITY OF BRITISH COLUMBIA, Vancouver (CA)

(72) Inventors: Michael Hayden, Vancouver (CA); Nicholas Caron, Vancouver (CA)

(21) Appl. No.: 19/105,298

(22) PCT Filed: Aug. 24, 2023
(86) PCT No.: PCT/CA2023/051123

§ 371 Date: Feb. 20, 2025

Related U.S. Application Data
(60) Provisional application No. 63/400,530, filed on Aug. 24, 2022.

(51) Int. Cl. G01N 33/68; G01N 33/74

(52) U.S. Cl. CPC G01N 33/6896; G01N 33/6848; G01N 33/74; G01N 2333/4716; G01N 2333/475; G01N 2333/65; G01N 2333/70; G01N 2333/765; G01N 2333/775; G01N 2333/90241; G01N 2333/96472; G01N 2800/2835; G01N 2800/52

(57) ABSTRACT

The present invention relates to biomarker-based analyses for the stratification of Huntington Disease (HD) in a subject. The invention further relates to protein biomarkers (and particular combinations) and their use in monitoring biochemical changes in HD patients indicative of the stage, severity, progression, or age-of-onset of disease; guidance for the design of clinical trials; for selecting a therapeutic regimen and monitoring response to treatment. The invention further comprises methods for the detection of HD biomarkers in cerebrospinal fluid (CSF) and other biofluids in patients with HD or at risk of developing HD.

### CROSS REFERENCE TO RELATED APPLICATIONS

This application claims the benefit of U.S. Provisional Patent Application Ser. No. 63/400,530 filed 24 Aug. 2022 entitled “CEREBROSPINAL FLUID BIOMARKERS FOR STRATIFICATION OF HUNTINGTON DISEASE AND USES THEREOF”.

### TECHNICAL FIELD

The present invention relates to biomarker-based analyses for the stratification of Huntington Disease (HD) in a subject. The invention further relates to protein biomarkers (and particular combinations) and their use in monitoring biochemical changes in HD patients indicative of the stage, severity, progression, or age-of-onset of disease; guidance for the design of clinical trials; for selecting a therapeutic regimen and monitoring response to treatment. The invention further comprises methods for the detection of HD biomarkers in cerebrospinal fluid (CSF) and other biofluids in patients with HD or at risk of developing HD.

### BACKGROUND

Huntington disease (HD) is an autosomal dominant neurodegenerative disease caused by a CAG expansion in the HTT gene that codes for an abnormal polyglutamine tract in the huntingtin protein (HTT).¹ Polyglutamine-expanded mutant huntingtin (mHTT), the primary pathogenic cause of HD, leads to the progressive loss of neuronal populations in the striatum as well as other structures in the basal ganglia and the cerebral cortex.²⁻⁷

HD typically manifests in the clinic as an adult-onset disease with affected individuals presenting with cognitive, motor and psychiatric disturbances.⁸ Prior to clinical diagnosis, there is a premanifest or prodromal stage of HD when cellular dysfunction and progressive neurodegeneration are occurring in the brain, but no overt symptoms are present. Age-of-onset, a timepoint when HD mutation carriers develop unequivocal motor signs of HD, is inversely correlated with CAG repeat length in expanded HTT,⁹ enabling broad predictions of disease onset.⁹ However, CAG repeat length only accounts for 50-60% of the variability,^{9,10} with other genetic and environmental factors reported to modify age-of-onset.¹¹⁻¹⁴

To date, there are no approved therapies to delay onset or slow progression of HD. Therapeutic approaches targeting the cause of HD, the CAG expanded HTT gene and its products, or downstream processes associated with the pathogenesis of HD, are currently in clinical development. Such therapies may be most effective if intervention is initiated prior to clinical onset and significant neurodegeneration in the brain. Two possible treatments in the clinical trial stage are AMT-130 and ANX005.^{82, 83, 84} AMT-130 is a double stranded RNA gene therapy designed to prevent the production of mutated huntingtin protein, via a single dose medication injected into the brain. ANX005 is an IV monoclonal antibody treatment intended to interfere with the immune system's complement pathway and ultimately slow down or prevent nerve damage in the brain, via a bi-monthly administration.

CSF is an accessible biofluid whose molecular composition reflects structural and functional changes in the brain, making it a promising biofluid for biomarker discovery in HD and other neurodegenerative disorders. In HD, biomarkers in CSF and other biofluids may offer the potential to monitor cell-type and/or pathway-specific pathophysiological alterations in the CNS over the natural history of disease. Sensitive biomarkers that reflect early cellular dysfunction or neurodegeneration in the brain during the premanifest stage of HD are needed to complement current predictive methods to improve accuracy of predictions of disease onset and guide the appropriate timing for therapeutic intervention. Moreover, such biomarkers are desired to complement existing clinical^{15,16} and imaging-based^{17,18} biomarkers for monitoring disease progression and assessing efficacy of candidate therapies in HD clinical trials.

Several promising molecular biomarkers have been identified in CSF and/or blood that are altered in HD^{19,20}. However, only mutant huntingtin (HTT) protein (mHTT)²¹⁻²⁵ and neurofilament light chain (NEFL or NfL)^{23,24,26-31} have been used in HD clinical trials.

CSF mHTT increases with disease progression²⁴ and its levels correlate with clinical measures of disease severity.²¹⁻²⁴ Importantly, a dose-dependent reduction of CSF mHTT was observed in a phase I/IIa clinical trial evaluating an intrathecally-delivered HTT-targeted antisense oligonucleotide (tominersen), suggesting that CSF mHTT could be a valuable biomarker to assess target engagement in the CNS.³² However, preliminary findings from the halted phase III trial evaluating the efficacy of tominersen (NCT03761849) suggest that a reduction of CSF mHTT alone may not predict clinical benefit.

NEFL in biofluids is a biomarker of neuronal injury, with elevated NEFL levels in CSF and blood reported in HD^{23,24,26-30} as well as other neurological diseases³³. In HD, NEFL levels in biofluids are correlated with clinical and imaging measures of disease^{23,26} and are a strong prognostic biomarker of disease onset, progression and brain atrophy in HD patients.^{24,26,28} Notably, NEFL is being used in HD clinical trials as an exploratory biomarker to monitor disease progression and to assess therapeutic efficacy. However, it remains unknown if NEFL in biofluids will respond to candidate therapies in a manner that is predictive of clinical benefit.

We analyzed a panel of pre-specified proteins in the CSF from manifest HD (manHD) patients, premanifest HD (preHD) and control individuals using nanoflow liquid chromatography-coupled parallel-reaction monitoring mass spectrometry (nanoLC-PRM-MS). This methodology can allow for the simultaneous identification and quantification of more than 30 peptides at attomole concentrations within a single run,³⁴⁻³⁶ allowing for reliable monitoring of CSF analytes with high specificity and sensitivity. An initial list of protein candidates was prioritized based on existing literature demonstrating altered levels in the CSF of HD mutation carriers, including complement C1q C chain (C1QC),³⁷ complement inhibitor C4b-binding protein (C4B),³⁷ chitinase-3-like 1 (CHI3L1) (also known as YKL-40),^{29,37,38} clusterin (CLU),^{39,40} cathepsin D (CTSD),³⁷ protocadherin Fat 2 (FAT2),⁴⁰ NEFL, prodynorphin (PDYN),⁴¹ proenkephalin (PENK),⁴⁰ and transthyretin (TTR).^{37,40,42} Additional protein candidates were selected that, to our knowledge, have not been previously measured in HD CSF, but were either reported to have altered expression in the striatum of HD patients,⁴³⁻⁴⁵ animal models of HD,^{46,47} or have been implicated in the pathogenesis of HD.^{48,49}

### SUMMARY

The present invention is based, in part, on the surprising discovery that a particular panel of biomarkers is especially useful for monitoring biochemical changes in HD patients indicative of the stage, severity, progression, or age-of-onset of disease; a role in guidance for the design of clinical trials; a role in selecting a therapeutic regimen and monitoring response to a treatment; a clinical role in narrowing or guiding treatment decisions, assigning a risk, making a diagnosis or confirming a clinical suspicion. Alternatively, the biomarkers as described herein, may be indicative of biochemical changes in HD. In particular, the biomarkers may be indicative of early biochemical changes underlying HD. Alternatively, the biomarkers may be useful following the transition from pre-manifest HD to manifest HD. Alternatively, the biomarkers may be useful for monitoring progression and/or severity of HD in subjects. The biomarker panels provide biomarkers which may be selected from one or more of: albumin (ALB); apolipoprotein E (APOE); brain-derived neurotrophic factor (BDNF); complement C1q B chain (C1QB); complement C1q C chain (C1QC); complement inhibitor C4b-binding protein (C4B); compliment C7 (C7); chitinase-3-like 1 (CHI3L1) (also known as YKL-40); clusterin (CLU); cannabinoid receptor 1 (CNR1); cathepsin D (CTSD); cytochrome C (CYCS); discoidin domain receptor family, member 1 (DRD1) also known as CD167a; discoidin domain receptor family, member 2 (DRD2) also known as CD167b; protocadherin Fat 2 (FAT2); D1 dopamine receptor-coupled protein, Golf (GNAL); indoleamine 2,3-dioxygenase 1 (IDO1); insulin-like growth factor 2 (IGF2); immunoglobulin heavy constant gamma 1 (IGHG1); neurofilament light chain (NEFL or NfL); cAMP and cAMP-inhibited cGMP 3′,5′-cyclic phosphodiesterase 10A (PDE10A); prodynorphin (PDYN); proenkephalin (PENK); protein phosphatase 1 regulatory subunit 1B (PPP1R1B) also known as dopamine- and cAMP-regulated neuronal phosphoprotein (DARPP-32); sigma-1 receptor (01R) (SIGMAR1); and transthyretin (TTR).

Herein we describe methods for HD stratification through detection of individual CSF proteins and combinations thereof of prioritized CSF protein biomarkers for classifying individuals based on HD mutation status and disease severity. Uses of HD biomarkers and HD stratification are also described.

In a first embodiment, there is provided a Huntington Disease (HD) biomarker panel, the panel including: (a) PENK alone; (b) PENK with NEFL; (c) PENK with IGHG1; (d) PENK with GNAL and IGHG1; (e) ALB alone; (f) APOE alone; (g) BDNF alone; (h) C7 alone; (i) CTSD alone; (j) DRD1 alone; (k) GNAL alone; (1) IDO1 alone; (m) IGF2 alone; (n) IGHG1 alone; (o) NEFL alone; (p) PDYN alone; or (q) combinations of any of the above; wherein the biomarker panel indicative of biochemical changes in HD.

In a further embodiment, there is provided a Huntington Disease (HD) biomarker panel, the panel including: PENK with NEFL; PENK with IGHG1; PENK with GNAL and IGHG1; ALB alone; APOE alone; BDNF alone; C7 alone; DRD1 alone; GNAL alone; IDO1 alone; IGF2 alone; IGHG1 alone; or combinations of any of the above; wherein the biomarker panel indicative of biochemical changes in HD.

In a further embodiment, there is provided a Huntington Disease (HD) biomarker panel, the panel including: PENK with NEFL; PENK with IGHG1; PENK with GNAL and IGHG1; APOE alone; C7 alone; GNAL alone; IDO1 alone; IGF2 alone; or combinations of any of the above; wherein the biomarker panel indicative of biochemical changes in HD.

In a further embodiment, there is provided a HD biomarker panel, the panel including: (a) ALB alone; (b) C4B alone; (c) IGHG1 alone; (d) TTR alone; (e) CNR1 alone; (f) PDYN alone; (g) PENK alone; (h) PPP1R1B alone; (i) APOE alone; (j) BDNF alone; (k) C1QB alone; (1) C7 alone; (m) FAT2 alone; (n) GNAL alone; (o) IGF2 alone; (p) NEFL alone; (q) PENK with ALB; (r) PENK with ALB and one of NEFL, IGF2, C7, BDNF, APOE, and IGHG1; (s) PENK with IGHG1 and NEFL; or (t) PENK with IGF2 and C7; wherein the biomarker panel is indicative of early biochemical changes in HD.

In a further embodiment, there is provided a HD biomarker panel, the panel including: ALB alone; IGHG1 alone; CNR1 alone; PPP1R1B alone; APOE alone; BDNF alone; C1QB alone; C7 alone; GNAL alone; IGF2 alone; PENK with ALB; PENK with ALB and one of NEFL, IGF2, C7, BDNF, APOE, and IGHG1; PENK with IGHG1 and NEFL; or PENK with IGF2 and C7; wherein the biomarker panel is indicative of early biochemical changes in HD.

In a further embodiment, there is provided a HD biomarker panel, the panel including: CNR1 alone; PPP1R1B alone; APOE alone; C1QB alone; C7 alone; GNAL alone; IGF2 alone; PENK with ALB; PENK with ALB and one of NEFL, IGF2, C7, BDNF, APOE, and
IGHG1; PENK with IGHG1 and NEFL; or PENK with IGF2 and C7; wherein the biomarker panel is indicative of early biochemical changes in HD.

In a further embodiment, there is provided a HD biomarker panel for predicting age of onset, the panel including: ALB alone; C4B alone; IGHG1 alone; TTR alone; APOE alone; BDNF alone; C1QB alone; C7 alone; FAT2 alone; GNAL alone; IGF2 alone; NEFL alone; and PENK with ALB.

In a further embodiment, there is provided a HD biomarker panel for monitoring early/mid HD to late HD, the panel including: CNR1 alone; PDYN alone; PENK alone; and PPP1R1B alone.

In a further embodiment, there is provided a HD biomarker panel the panel including: PENK with ALB and one of NEFL, IGF2, C7, BDNF, APOE, and IGHG1; PENK with IGHG1 and NEFL; or PENK with IGF2 and C7; wherein the biomarker panel is indicative of early biochemical changes in HD.

In a further embodiment, there is provided a HD biomarker panel, the panel including: (a) CHI3L1 alone; (b) C4B with IGHG1; (c) C4B, IGHG1, and NEFL; (d) CHI3L1 with C4B, IGHG1, and ALB; (e) PPP1R1B alone; (f) PPP1R1B with TTR; (g) TTR and CHI3L1 with one of: CTSD; and PPP1R1B; (h) C4B, TTR, and CNR1; (i) PPP1R1B, TTR, CTSD with one of: CHI3L1; ALB; C4B; and C1QB; (j) TTR, CHI3L1, and CTSD with one of: ALB; CYCS; CNR1; and C1QB; (k) TTR, ALB, and CYCS with one of: C4B; and PPP1R1B; (1) TTR, CTSD, and CYCS with one of: ALB; and C1QB; (m) C4B, TTR, CTSD, and CNR1; (n) C1QB, TTR, CTSD, and CNR1; (o) C1QB alone; (p) C4B alone; or (q) TTR alone; wherein the biomarker panel is for following the transition from pre-manifest HD to manifest HD.

In a further embodiment, there is provided a HD biomarker panel, the panel including: C4B with IGHG1; C4B, IGHG1, and NEFL; CHI3L1 with C4B, IGHG1, and ALB; PPP1R1B alone; PPP1R1B with TTR; TTR and CHI3L1 with one of: CTSD; and PPP1R1B; C4B, TTR, and CNR1; PPP1R1B, TTR, CTSD with one of: CHI3L1; ALB; C4B; and C1QB; TTR, CHI3L1, and CTSD with one of: ALB; CYCS; CNR1; and C1QB; TTR, ALB, and CYCS with one of: C4B; and PPP1R1B; TTR, CTSD, and CYCS with one of: ALB; and C1QB; C4B, TTR, CTSD, and CNR1; C1QB, TTR, CTSD, and CNR1; C1QB alone; or wherein the biomarker panel is for following the transition from pre-manifest HD to manifest HD.

In a further embodiment, there is provided a HD biomarker panel, the panel including: C4B with IGHG1; C4B, IGHG1, and NEFL; CHI3L1 with C4B, IGHG1, and ALB; PPP1R1B with TTR; TTR and CHI3L1 with one of: CTSD; and PPP1R1B; C4B, TTR, and CNR1; PPP1R1B, TTR, CTSD with one of: CHI3L1; ALB; C4B; and C1QB; TTR, CHI3L1, and CTSD with one of: ALB; CYCS; CNR1; and C1QB; TTR, ALB, and CYCS with one of: C4B; and PPP1R1B; TTR, CTSD, and CYCS with one of: ALB; and C1QB; C4B, TTR, CTSD, and CNR1; C1QB, TTR, CTSD, and CNR1; wherein the biomarker panel is for following the transition from pre-manifest HD to manifest HD.

In a further embodiment, there is provided a HD biomarker panel, the panel including: PPP1R1B alone; or C1QB alone; wherein the biomarker panel is for following the transition from pre-manifest HD to manifest HD.

In a further embodiment, there is provided a HD biomarker panel for monitoring pre HD to manifest HD, the panel including: CHI3L1 alone; C4B with IGHG1; C4B, IGHG1, and NEFL; or CHI3L1 with C4B, IGHG1, and ALB,

In a further embodiment, there is provided a HD biomarker panel for monitoring pre HD to early/mid HD, the panel including: PPP1R1B alone; PPP1R1B with TTR; TTR and CHI3L1 with one of: CTSD; and PPP1R1B; C4B, TTR, and CNR1; PPP1R1B, TTR, CTSD with one of: CHI3L1; ALB; C4B; and C1QB; TTR, CHI3L1, and CTSD with one of: ALB; CYCS; CNR1; and C1QB; TTR, ALB, and CYCS with one of: C4B; and PPP1R1B; TTR, CTSD, and CYCS with one of: ALB; and C1QB; C4B, TTR, CTSD, and CNR1; or C1QB, TTR, CTSD, and CNR1.

In a further embodiment, there is provided a HD biomarker panel for monitoring pre HD to early/mid HD, the panel including: C1QB alone; C4B alone; or TTR alone; wherein the biomarker panel is for following the transition from pre-manifest HD to manifest HD.

In a further embodiment, there is provided a HD biomarker panel, the panel including: (a) PDYN; (b) PDYN with PENK; (c) PDYN and IGHG1 with one of: PENK; and C1QB; (d) PENK, CNR1, and IGF2; (e) CNR1, C1QB, and IGHG1; (f) CNR1, PPP1R1B, APOE, and IGHG1; (g) CNR1, PPP1R1B, BDNF; APOE, and IGHG1; (h) CNR1, BDNF; APOE, IGF2, and IDO1; (i) CNR1, BDNF; C1QB, IGF2, and IDO1; (j) CNR1, PPP1R1B, BDNF; C1QB, and IGHG1; (k) PDYN, CNR1, PPP1R1B, C1QB, and IGHG1; (I) APOE; (m) BDNF; (n) C1QB; (o) CNR1; (p) IDO1; (q) IGF2; (r) IGHG1; (s) NEFL; (t) PENK; (u) PP1R1B; or (v) TTR; wherein the biomarker panel is for monitoring progression of HD or severity of HD or progression and severity of HD.

In a further embodiment, there is provided a HD biomarker panel, the panel including: PDYN with PENK; PDYN and IGHG1 with one of: PENK; and C1QB; PENK, CNR1, and IGF2; CNR1, C1QB, and IGHG1; CNR1, PPP1R1B, APOE, and IGHG1; CNR1, PPP1R1B, BDNF; APOE, and IGHG1; CNR1, BDNF; APOE, IGF2, and IDO1; CNR1, BDNF; C1QB, IGF2, and IDO1; CNR1, PPP1R1B, BDNF; C1QB, and IGHG1; PDYN, CNR1, PPP1R1B, C1QB, and IGHG1; APOE; BDNF; C1QB; CNR1; IDO1; IGF2; IGHG1; or PP1R1B; wherein the biomarker panel is for monitoring progression of HD or severity of HD or progression and severity of HD.

In a further embodiment, there is provided a HD biomarker panel, the panel including: PDYN with PENK; PDYN and IGHG1 with one of: PENK; and C1QB; PENK, CNR1, and IGF2; CNR1, C1QB, and IGHG1; CNR1, PPP1R1B, APOE, and IGHG1; CNR1, PPP1R1B, BDNF; APOE, and IGHG1; CNR1, BDNF; APOE, IGF2, and IDO1; CNR1, BDNF; C1QB, IGF2, and IDO1; CNR1, PPP1R1B, BDNF; C1QB, and IGHG1; PDYN, CNR1, PPP1R1B, C1QB, and IGHG1; APOE; C1QB; CNR1; IDO1; IGF2; or PP1R1B; wherein the biomarker panel is for monitoring progression of HD or severity of HD or progression and severity of HD.

In a further embodiment, there is provided a HD biomarker panel, the panel including: PDYN; PDYN with PENK; PDYN and IGHG1 with one of: PENK; and C1QB; PENK, CNR1, and IGF2; CNR1, C1QB, and IGHG1; CNR1, PPP1R1B, APOE, and IGHG1; CNR1, PPP1R1B, BDNF; APOE, and IGHG1; CNR1, BDNF; APOE, IGF2, and IDO1; CNR1, BDNF; C1QB, IGF2, and IDO1; CNR1, PPP1R1B, BDNF; C1QB, and IGHG1; or PDYN, CNR1, PPP1R1B, C1QB, and IGHG1; wherein the biomarker panel is for monitoring progression of HD or severity of HD or progression and severity of HD comparing early/mid HD and late HD.

In a further embodiment, there is provided a HD biomarker panel, the panel including: APOE; BDNF; C1QB; CNR1; IDO1; IGF2; IGHG1; NEFL; PENK; PP1R1B; or TTR; wherein the biomarker panel is for monitoring progression of HD or severity of HD or progression and severity of HD comparing early/mid HD and late HD.

The one or more members of the biomarker panel may be indicative of biochemical changes in HD in a patient. The one or more members of the biomarker panel may be indicative of early biochemical changes in HD in a patient. The one or more members of the biomarker panel may be indicative of the transition from pre-manifest HD to manifest HD in a patient. The one or more members of the biomarker panel may be for for monitoring progression of HD or severity of HD or progression and severity of HD in a patient.

The HD biomarker panel described herein may further include one or more clinical measures of disease severity selected from the following: (i) a composite Unified Huntington's Disease Rating Scale (cUHDRS); (ii) a Stroop word reading (SWR); (iii) a symbol digit modality test (SDMT); (iv) a total functional capacity (TFC); (v) a total motor score (TMS); and (vi) a Q-motor score. The HD biomarker panel described herein may further include cUHDRS score. The HD biomarker panel described herein may further include SWR. The HD biomarker panel described herein may further include SDMT. The HD biomarker panel described herein may further include TFC. The HD biomarker panel described herein may further include TMS. The HD biomarker panel described herein may further include Q-motor score.

The HD biomarker panel may be determined from a biological sample obtained from a human subject. The biological sample may be selected from the group consisting of: whole blood; blood plasma; blood serum; and cerebrospinal fluid (CSF). The biological sample may be CSF.

The HD biomarker panel as described herein, wherein the biomarker is selected from one or more of: NEFL; GNAL; DRD1; IGF2; IGHG1; CHI3L1; C7; FAT2; ALB; and C4B and wherein the one or more biomarkers show an increase in a cerebrospinal fluid (CSF) sample from a subject in comparison to a non-HD standard amount of protein may be useful as an indicator of HD severity and/or progression; or wherein the biomarker is selected from one or more of: TTR; IDO1; CNR1; CTSD; C1QB; PPP1R1B; APOE; BDNF; PDYN; and PENK and wherein the one or more biomarkers show a decrease in a CSF sample from a subject in comparison to a non-HD standard amount of protein may be useful as an indicator of HD severity and/or progression.

The HD biomarker panel may be used to detect a target protein or a target peptide in a biological sample using a mass spectrometry assay or an immunoassay. The mass spectrometry assay may be selected from one or more of: a nanoflow liquid chromatography-coupled parallel-reaction monitoring mass spectrometry (nanoLC-PRM-MS); gas chromatography coupled to mass spectrometry (GC-MS); liquid chromatography with mass spectrometry (LC-MS); electrospray ionization (ESI); matrix-assisted laser desorption/ionization (MALDI); matrix assisted laser desorption ionization-time of flight mass spectrometry (MALDI-TOF-MS); and liquid chromatography tandem mass spectrometry (LC-MS/MS). The immunoassay may be an enzyme linked immunoassay (ELISA) or an enzyme multiplied immunoassay technique (EMIT). Alternatively, surface plasmon resonance (SPR) may be used to detect a target protein or a target peptide in a biological sample.

In a further embodiment, there is provided a method of detecting the level of a biomarker panel in a subject suspected of having HD or known to have HD, the method including: (a) measuring, in a biological sample obtained from the subject, a relative concentration of at least one biomarker in the biomarker panel, wherein the biomarker panel is set out herein; and (b) monitoring biochemical changes in HD, monitoring biochemical changes in early HD, monitoring progression of HD, or monitoring the severity of HD.

In a further embodiment, there is provided a method of detecting the level of a biomarker panel in a subject suspected of having HD or known to have HD, the method including: (a) measuring, in a biological sample obtained from the subject, a relative concentration of at least one biomarker in the biomarker panel, wherein the biomarker panel is set out herein; and (b) monitoring biochemical changes in HD, monitoring monitoring biochemical changes in HD patients indicative of the stage, severity, progression, or age-of-onset of HD; a role in guidance for the design of clinical trials; a role in selecting a therapeutic regimen and monitoring response to a treatment; a clinical role in narrowing or guiding treatment decisions, assigning a risk, making a diagnosis or confirming a clinical suspicion. Biomarkers as described herein, may be indicative of biochemical changes in HD. In particular, the biomarkers may be indicative of early biochemical changes underlying HD. Alternatively, the biomarkers may be useful following the transition from pre-manifest HD to manifest HD. Alternatively, the biomarkers may be useful for monitoring progression and/or severity of HD in subjects.

The biological sample may be selected from: CSF, whole blood; blood serum; and blood plasma. The biological sample may be CSF. The subject may be a human. The subject may have been determined to have a CAG repeat expansion mutation in HTT.

The subject may be assessed for biochemical changes in HD and wherein the biomarker panel is set out herein and where the biological sample has a: (a) decreased PENK; (b) decreased PENK and increased NEFL; (c) decreased PENK and increased IGHG1; (d) decreased PENK, increased GNAL, and increased IGHG1; (e) increased ALB; (f) decreased APOE; (g) decreased BDNF; (h) increased C7; (i) decreased CTSD; (j) increased DRD1; (k) increased GNAL; (1) decreased IDO1; (m) increased IGF2; (n) increased IGHG1; (o) increased NEFL; (p) decreased PDYN; or (q) combinations of any of the above; as compared to a control non-HD or to a non-HD standard at an approximately equivalent age.

The subject may be assessed for biochemical changes in HD and wherein the biomarker panel is set out herein and where the biological sample may have a: (a) decreased PENK; (b) decreased PENK and increased NEFL; (c) decreased PENK and increased IGHG1; (d) decreased PENK, increased GNAL, and increased IGHG1; (e) increased ALB; (f) decreased APOE; (g) decreased BDNF; (h) increased C7; (i) decreased CTSD; (j) increased DRD1; (k) increased GNAL; (1) decreased IDO1; (m) increased IGF2; (n) increased IGHG1; (o) increased NEFL; (p) decreased PDYN; or (q) combinations of any of the above; when compared to a control non-HD sample or to a non-HD standard at an approximately equivalent age as an indication of HD or preHD status.

The subject may be assessed for early biochemical changes in HD and wherein the biomarker panel is set out herein and where the biological sample may have a: (a) increased ALB; (b) increased C4B; (c) increased IGHG1; (d) decreased TTR; (e) decreased CNR1; (f) decreased PDYN; (g) decreased PENK; (h) decreased PPP1R1B; (i) decreased APOE; (j) decreased BDNF; (k) decreased C1QB; (1) increased C7; (m) increased FAT2; (n) increased GNAL; (o) increased IGF2; (p) increased NEFL; (q) decreased PENK, increased ALB; (r) decreased PENK, increased ALB and one of increased NEFL, increased IGF2, increased C7, decreased BDNF, decreased APOE, and increased IGHG1; (s) decreased PENK, increased IGHG1 and increased NEFL; or (t) decreased PENK, increased IGF2 and increased C7; as compared to a control non-HD or to a non-HD standard at an approximately equivalent age.

The subject may be assessed for age of onset of HD and wherein the biomarker panel is set out herein and where the biological sample may have a: increased ALB; increased C4B; increased IGHG1; decreased TTR; decreased APOE; decreased BDNF; decreased C1QB; increased C7; increased FAT2; increased GNAL; increased IGF2; increased NEFL; or decreased PENK, increased ALB; as compared to a control non-HD or to a non-HD standard at an approximately equivalent age.

The subject may be assessed for the stage of HD and wherein the biomarker panel is set out herein and where the biological sample may have a: decreased CNR1; decreased PDYN; decreased PENK; decreased PPP1R1B; as compared to a control non-HD or to a non-HD standard at an approximately equivalent age.

The subject may be assessed for early biochemical changes in HD and wherein the biomarker panel is set out herein and where the biological sample may have a: decreased PENK, increased ALB and one of increased NEFL, increased IGF2, increased C7, decreased BDNF, decreased APOE, and increased IGHG1; decreased PENK, increased IGHG1 and increased NEFL; or decreased PENK, increased IGF2 and increased C7; as compared to a control non-HD or to a non-HD standard at an approximately equivalent age.

The subject may be assessed for the transition from pre-manifest HD to manifest HD and wherein the biomarker panel is set out herein and where the biological sample may have a: (a) increased CHI3L1; (b) increased C4B and increased IGHG1; (c) increased C4B, increased IGHG1, and increased NEFL; (d) increased CHI3L1, increased C4B, increased IGHG1, and decreased ALB; (e) increased PPP1R1B; (f) increased PPP1R1B and increased TTR; (g) increased TTR, increased CHI3L1, and one of: decreased CTSD; and increased PPP1R1B; (h) increased C4B, increased TTR, and decreased CNR1; (i) increased PPP1R1B, increased TTR, decreased CTSD and one of: increased CHI3L1; decreased ALB; increased C4B; and increased C1QB; (j) increased TTR, increased CHI3L1, decreased CTSD, and one of: decreased ALB; increased CYCS; decreased CNR1; and increased C1QB; (k) increased TTR, decreased ALB, increased CYCS, and one of: increased C4B; and increased PPP1R1B; (1) increased TTR, decreased CTSD, increased CYCS, and one of: decreased ALB; and increased C1QB; (m) increased C4B, increased TTR, decreased CTSD, and decreased CNR1; (n) increased C1QB, increased TTR, decreased CTSD, and decreased CNR1; (o) increased C1QB; (p) increased C4B; or (q) increased TTR; when manifest HD is compared to pre-manifest HD at an equivalent age.

The subject may be assessed for the transition from pre-manifest HD to manifest HD and wherein the biomarker panel is set out herein and where the biological sample may have a: increased CHI3L1; increased C4B and increased IGHG1; increased C4B, increased IGHG1, and increased NEFL; or increased CHI3L1, increased C4B, increased IGHG1, and decreased ALB; when manifest HD is compared to pre-manifest HD at an equivalent age.

The subject may be assessed for the transition from pre-manifest HD to early/mid HD and wherein the biomarker panel is set out herein and where the biological sample may have a: increased PPP1R1B; increased PPP1R1B and increased TTR; increased TTR, increased CHI3L1, and one of: decreased CTSD; and increased PPP1R1B; increased C4B, increased TTR, and decreased CNR1; increased PPP1R1B, increased TTR, decreased CTSD and one of: increased CHI3L1; decreased ALB; increased C4B; and increased C1QB; increased TTR, increased CHI3L1, decreased CTSD, and one of: decreased ALB; increased CYCS; decreased CNR1; and increased C1QB; increased TTR, decreased ALB, increased CYCS, and one of: increased C4B; and increased PPP1R1B; increased TTR, decreased CTSD, increased CYCS, and one of: decreased ALB; and increased C1QB; increased C4B, increased TTR, decreased CTSD, and decreased CNR1; or increased C1QB, increased TTR, decreased CTSD, and decreased CNR1; when early/mid HD is compared to pre-manifest HD at an equivalent age.

The subject may be assessed for the transition from pre-manifest HD to manifest HD and wherein the biomarker panel is set out herein and where the biological sample may have a: increased C1QB; increased C4B; or increased TTR; when manifest HD is compared to pre-manifest HD at an equivalent age.

The subject may be assessed for for monitoring progression of HD or severity of HD or progression and severity of HD and wherein the biomarker panel is set out herein and where the biological sample may have a: (a) decreased PDYN; (b) decreased PDYN, and decreased PENK; (c) decreased PDYN, and increased IGHG1 and one of: decreased PENK; and decreased C1QB; (d) decreased PENK, decreased CNR1, and increased IGF2; (e) decreased CNR1, decreased C1QB, and increased IGHG1; (f) decreased CNR1, decreased PPP1R1B, decreased APOE, and increased IGHG1; (g) decreased CNR1, decreased PPP1R1B, decreased BDNF; decreased APOE, and increased IGHG1; (h) decreased CNR1, decreased BDNF; decreased APOE, increased IGF2, and decreased IDO1; (i) decreased CNR1, decreased BDNF; decreased C1QB, increased IGF2, and decreased IDO1; (j) decreased CNR1, decreased PPP1R1B, decreased BDNF; decreased C1QB, and increased IGHG1; or (k) decreased PDYN, decreased CNR1, decreased PPP1R1B, decreased C1QB, and increased IGHG1; (1) decreased APOE; (m) decreased BDNF; (n) decreased C1QB; (o) decreased CNR1; (p) decreased IDO1; (q) increased IGF2; (r) increased IGHG1; (s) increased NEFL; (t) decreased PENK; (u) decreased PP1R1B; or (v) decreased TTR; when late HD is compared to early/mid HD at an equivalent age.

The subject known to have HD may be further administered an HD treatment. The subject known to have HD may be further administered an HD treatment and the subject may be further monitored for their response to the HD treatment based on biochemical changes as determined by testing of one or more of the biomarker panels described herein. The HD treatment may be selected from one or more of: an antisense oligonucleotide, a siRNA, a miRNA, a small molecule, a CRISPR gene edit, wherein the HD treatment lowers levels of the mutant HTT protein in the CNS. The HD treatment may be selected from one or more of: tominersen, AMT-130, or ANX005. The HD treatment may be selected from one or more of: AMT-130, or ANX005.

In a further embodiment, there is provided a method for monitoring response to treatment of HD and determining treatment efficacy in a subject, including the steps of: (a) measuring levels of at least one biomarker in at least 2 longitudinal biological samples from the same subject and comparing the measured levels to an level of a matched biomarker determined in a clinically relevant population, wherein the at least one biomarker is from a first panel, comprising: NEFL; GNAL; DRD1; IGF2; IGHG1; CHI3L1; C7; FAT2; ALB; PDE10A; CLU; C4B; CYCS; DRD2; SIGMAR1; TTR; Q1QC; IDO1; CNR1; CTSD; C1QB; PPP1R1B; APOE; BDNF; PDYN; and PENK, wherein the level of the one or more biomarkers in the biological sample is changed, and wherein at least one of the at least two biological samples is collected before the individual is treated for HD and at least one of the at least two biological samples is collected after the subject is treated for HD; (b) calculating a score for the at least one biomarker in the biological samples, by summing: the number of biomarkers in the first panel exhibiting a change in level relative to the of the biomarker determined in a clinically relevant population, and/or the number of biomarkers in the second panel exhibiting a change in level relative to the of the biomarker determined in a clinically relevant population; and (c) determining that said treatment(s) is effective if the score of the panel of biomarker(s) in the sample collected after treatment is lower than the score of at least one of the at least two biologicals samples collected before treatment.

The at least one biomarker may be from a panel, including: NEFL; GNAL; DRD1; IGF2; IGHG1; CHI3L1; C7; FAT2; ALB; PDE10A; CLU; C4B; CYCS; TTR; Q1QC; IDO1; CNR1; CTSD; C1QB; PPP1R1B; APOE; BDNF; PDYN; and PENK.

In another aspect, there is provided, a method is provided for the stratification of HD, the method comprising:
a) obtaining a sample of body fluid from HD mutation carriers (including preHD, early/mid HD and late HD individuals) and determining the levels of protein biomarkers in the sample;
b) obtaining a sample of biofluid from a control individual (not carrying the HD mutation) and determining the levels of protein biomarkers in the sample;
c) detecting altered levels of protein biomarkers in the HD mutation carriers compared to controls; preHD compared to controls; manHD compared to preHD; early/midHD compared to preHD and lateHD compared to early/mid HD;
   wherein the alterations are indicative of an individual being an HD mutation carrier, premanifest HD, manifest HD, early/mid HD or late HD. Biofluids are preferably cerebrospinal fluid (CSF) but may also include blood.

In another aspect of the invention are protein biomarkers with altered levels in HD mutation carriers compared to control individuals who do not carry the HD mutation. The biomarkers may comprise single or preferred combinations of proteins including;
a) PENK alone or
b) in combination with one or more of NEFL; IGHG1 or GNAL.

In another aspect, there are provided, protein biomarkers with altered levels in premanifest HD compared to control individuals who do not carry the HD mutation. The biomarkers may comprise single or preferred combinations of proteins including;
a) PENK alone or
b) in combination with one or more of ALB; NEFL; IGF2; C7; BDNF; APOE or IGHG1.

In another aspect, there are provided protein biomarkers with altered levels in manifest HD individuals compared to premanifest HD individuals, indicative of disease progression. The biomarkers may comprise single or preferred combinations of proteins including;
a) CHI3L1 alone or
b) combinations of two or more of CHI3L1; C4B; IGHG1; NEFL or ALB.

In another aspect, there are provided protein biomarkers with altered levels in early/mid HD individuals compared to premanifest HD individuals, indicative of disease progression. The biomarkers may comprise single or preferred combinations of proteins including;
a) PPP1R1B alone or
b) combinations with two or more of PPP1R1B; TTR; CHI3L1; CTSD; C4B; CNR1; CTSD; ALB; CYCS; C1QB

In another aspect, there are provided protein biomarkers with altered levels in late HD individuals compared to early/mid HD individuals, indicative of disease progression. The biomarkers may comprise single or preferred combinations of proteins including;
a) PDYN alone or
b) PDYN in combination with one or more of PENK; IGHG1; C1QB; CNR1 or PPP1R1B;
c) CNR1 in combination with two or more of PENK; IGF2; C1QB; IGHG1; PPP1R1B; APOE; BDNF; IDO1 or PDYN

In another aspect, there are provided methods for predicting age-of-onset of HD comprising detection of biomarkers in individuals determined to be in premanifest HD. In another aspect, there are provided methods for improving the accuracy of age-of-onset of HD as determined by the length of CAG repeats in the HD mutation comprising detection of biomarkers in individuals determined to be in premanifest HD in addition to determination of CAG repeat length. The biomarkers may comprise single or preferred combinations of proteins including;
a) ALB, C4B, IGHG1 and TTR1 as single biomarkers or
b) a combination of two or more of said markers in the above (a), wherein the detection of said biomarkers may improve the accuracy of predictions of age-of-onset by the following degrees;
a) within 10-15 years of preHD biomarker detection;
b) within 5-10 years of preHD biomarker detection or
c) within <5 years of preHD biomarker detection.

In another aspect, there are provided a method for determining a more accurate (early or late) age-of-onset method for optimal timing of therapeutic regimen. By way of example, for individuals in which premanifest HD biomarkers (singly or in combinations) predict an early age-of-onset initiation of treatment with inhibitors of HD progression or neuroprotective drugs could be recommended before clinical onset of the disease. Drugs that inhibit HD progression or have neuroprotective effect may include those that either influence pathways that are altered before the clinical manifestation of the disease (eg BDNF secretion) or mitochondrial function or by directly targeting the cause for HD by lowering the level of the mutant gene product.

In another aspect, there are provided methods for guiding the design of clinical trials or for monitoring efficacy of novel HD therapeutics comprising detection of said biomarkers or combinations thereof to stratify individuals involved in clinical trials according to stage of HD.

In another aspect, there are provided methods for detecting such biomarkers (or combinations thereof). Methods of detection may include, but are not limited to, assays such as mass spectrometry (preferably nanoflow liquid chromatography-coupled parallel-reaction monitoring mass spectrometry (nanoLC-PRM-MS)) and immunoassays (preferably enzyme linked immunoassay-ELISA).

### BRIEF DESCRIPTIONS OF THE DRAWINGS

FIG. 1: Comparison of CSF protein levels across disease stages. Box and whisker plots comparing normalized CSF protein levels (values represent arbitrary units, a.u.) between controls (n=8), preHD (n=8), early/mid HD (TFC>5; n=8), and late HD (TFC<5; n=8) individuals. Intergroup differences were assessed using ANCOVA including age as a covariate and summary statistics are shown at the top of each plot. Post hoc tests were performed using Tukey's test to correct for multiple comparisons. (A) C1QB (*P=0.010 compared to controls, ##P=0.008 compared to early/mid HD), (B) CNR1 (##P=0.008 compared to early/mid HD), (C) IDO1 (*P=0.020 compared to controls), (D) IGF2 (*P=0.012 compared to controls), (E) IGHG1 (** P=0.004 compared to controls), (F) NEFL (** P=0.002 compared to controls), (G) PDYN (*P=0.012, ** P=0.004, *** P=0.0003 compared to controls) and (H) PENK (*P=0.012, ** P=0.004, *** P=0.0002 compared to controls). Individual data points are plotted for each group. Boxes show 25th to 75th percentiles, the central band denotes the median, the plus sign denotes the mean, and the whiskers show the minimum and maximum values. [a.u.=arbitrary units; preHD=premanifest HD]

FIG. 2: Relative importance of CSF protein markers from discriminating between HD stages. CSF proteins ranked based on their relative importance for discriminating (A) HD mutation carriers from controls, (B) preHD from controls and (C) manHD from preHD groups. Fold changes represent the ratio of age-adjusted means between groups where colour scales were added to highlight increased (shaded) or decreased (shaded) levels in each comparison. Grey bars represent the percent (%) contribution of each variable to the bootstrapped sPLS-DA model. Age-adjusted values were used for all analyses.

FIG. 3: Exploratory multi-marker CSF protein panels for discriminating subjects based on HD mutation status and disease severity. ROC curve analysis showing AUC values of individual and combinations of CSF proteins with the highest discriminatory ability for distinguishing (A) HD mutation carriers from controls, (B) preHD from controls, (C) manHD from preHD, (D) early/mid HD from preHD, and (E) late HD from early/mid HD.

FIG. 4: Comparison of NEFL and PENK discriminatory ability for distinguishing HD mutation carriers from controls. ROC curve and AUC values comparing the discriminatory ability of NEFL and PENK for distinguishing between HD mutation carriers and controls.

FIG. 5: Peptide sequences and correlations between individual peptides measured by nanoLC-PRM-MS. Table summarizing peptide sequences measured by nanoLC-PRM-MS and correlations between unadjusted peptide values for each of the 26 CSF protein candidates in all participants. Pearson's correlation coefficients (r) are presented and shaded scales were added to highlight positive correlations.

FIG. 6: Comparison of CSF protein levels across disease stages. Fold changes and comparisons of age-adjusted CSF protein levels between controls, preHD, early/mid HD, and late HD individuals. Intergroup differences were assessed using ANCOVA including age as a covariate followed by posthoc analysis using Tukey's test to correct for multiple comparisons. Fold changes represent the ratio of age-adjusted means between groups. shaded areas were added to highlight increased or decreased levels for each comparison. P-values<0.05 are shown in bold. *Candidates not previously investigated in HD CSF.

FIG. 7: Correlations between CSF proteins in HD mutation carriers. Associations between age-adjusted CSF protein analyte levels was performed in all HD mutation carriers. Correlations were assessed using Pearson's partial correlation including age as a covariate. Analytes were grouped according to their association with biological processes related to neuronal function, motor behaviour, cognition and memory, synapse organization and plasticity, complement pathway activation, immune response, and apoptosis and cell death. Functional enrichment analysis of all CSF proteins was performed using g: GOSt (gProfiler™). Pearson's correlation coefficients (r) are presented and shaded scales were added to highlight positive and negative correlations. Correlations with P-values<0.05 are shown in bold.

FIG. 8: Discriminatory power of individual CSF markers for distinguishing between disease stages. ROC curve analysis evaluating the discriminatory performance of each individual CSF protein for distinguishing subjects based on HD mutation status and disease severity. Age-adjusted values were used to generate ROC curves. P-values<0.05 are shown in bold. P-values presented were not corrected for multiple comparisons.

FIG. 9: Correlation of CSF proteins with predicted age of onset. Pearson's correlation coefficients and p-values for relationship of predicted age-of-onset with unadjusted CSF protein values.

### DETAILED DESCRIPTION

The following detailed description will be better understood when read in conjunction with the appended figures. For the purpose of illustrating the invention, the figures demonstrate embodiments of the present invention. However, the invention is not limited to the precise arrangements, examples, and instrumentalities shown. Any terms not directly defined herein shall be understood to have the meanings commonly associated with them as understood within the art of the invention.

### Definitions

Any terms not directly defined herein shall be understood to have the meanings commonly associated with them as understood within the art of the invention.

As used herein the term “biomarker” or “biological marker” is any measurable indicator of a biological state. Biomarkers as used herein may be protein analytes. These biomarkers may have a role in monitoring biochemical changes in HD patients indicative of the stage, severity, progression, or age-of-onset of disease; a role in guidance for the design of clinical trials; a role in selecting a therapeutic regimen and monitoring response to a treatment; a clinical role in narrowing or guiding treatment decisions, assigning a risk, making a diagnosis or confirming a clinical suspicion. Biomarkers as described herein, may be indicative of biochemical changes in HD. In particular, the biomarkers may be indicative of early biochemical changes underlying HD. Alternatively, the biomarkers may be useful following the transition from pre-manifest HD to manifest HD. Alternatively, the biomarkers may be useful for monitoring progression and/or severity of HD in subjects.

As used herein the term “an HD biomarker panel” is meant to include a collection of individual biomarkers used either alone or in combination as indicative of biochemical changes in HD; indicative of early biochemical changes underlying HD; or for monitoring progression and/or severity of HD.

As used herein a “biomarker” or a “biological marker” may be selected from one or more of: albumin (ALB); apolipoprotein E (APOE); brain-derived neurotrophic factor (BDNF); complement C1q B chain (C1QB); complement C1q C chain (C1QC); complement inhibitor C4b-binding protein (C4B); compliment C7 (C7); chitinase-3-like 1 (CHI3L1) (also known as YKL-40); clusterin (CLU); cannabinoid receptor 1 (CNR1); cathepsin D (CTSD); cytochrome C (CYCS); discoidin domain receptor family, member 1 (DRD1) also known as CD167a; discoidin domain receptor family, member 2 (DRD2) also known as CD167b; protocadherin Fat 2 (FAT2); D1 dopamine receptor-coupled protein, Golf (GNAL); indoleamine 2,3-dioxygenase 1 (IDO1); insulin-like growth factor 2 (IGF2); immunoglobulin heavy constant gamma 1 (IGHG1); neurofilament light chain (NEFL or NfL); CAMP and cAMP-inhibited cGMP 3′,5′-cyclic phosphodiesterase 10A (PDE10A); prodynorphin (PDYN); proenkephalin (PENK); protein phosphatase 1 regulatory subunit 1B (PPP1R1B) also known as dopamine- and cAMP-regulated neuronal phosphoprotein (DARPP-32); sigma-1 receptor (01R) (SIGMAR1); and transthyretin (TTR). Alternatively, a “biomarker” or a “biological marker” may be selected from one or more of: NEFL; GNAL; DRD1; IGF2; IGHG1; CHI3L1; C7; FAT2; ALB; PDE10A; CLU; C4B; CYCS; TTR; Q1QC; ID01; CNR1; CTSD; C1QB; PPP1R1B; APOE; BDNF; PDYN; and PENK. As disclosed herein, depending on the HD patient groups being compared different biomarkers are have different significance and will either increase or decrease depending on the circumstances. For example, PP1R1B would be expected to be decreased in late HD as compared to early/mid HD at an equivalent age, but when comparing manifest HD to premanifest HD at an equivalent age PPP1R1B would be expected to be increased.

Generally, an area under the curve (AUC) of 0.7 or greater is a good indicator of a biomarker's relevance as a marker for biochemical changes in an HD patient's sample. Alternatively, an AUC of 0.69 or greater is a good indicator of a biomarker's relevance as a marker for biochemical changes in an HD patient's sample.

Alternatively, the term “HD biomarker panel” is meant to include: (a) PENK alone; (b) PENK with NEFL; (c) PENK with immunoglobulin heavy constant gamma 1 (IGHG1); (d) PENK with D1 dopamine receptor-coupled protein, Golf (GNAL) and IGHG1; (e) PENK with albumin (ALB); (f) PENK with ALB and one of NEFL, insulin-like growth factor 2 (IGF2), compliment C7 (C7), brain-derived neurotrophic factor (BDNF), apolipoprotein E (APOE), and IGHG1; (e) PENK with IGHG1 and NEFL; or (f) PENK with IGF2 and C7; wherein the biomarker panel indicative of biochemical changes in HD.

Alternatively, the term “HD biomarker panel” is meant to include: (a) ALB alone; (b) C4B alone; (c) IGHG1 alone; (d) TTR alone; (e) cannabinoid receptor 1 (CNR1) alone; (f) PDYN alone; (g) PENK alone; or (h) protein phosphatase 1 regulatory subunit 1B (PPP1R1B) alone; wherein the biomarker panel is indicative of early biochemical changes underlying HD.

Alternatively, the term “HD biomarker panel” is meant to include: (a) CHI3L1 alone; (b) C4B with IGHG1; (c) C4B, IGHG1, and NEFL; (d) CHI3L1 with C4B, IGHG1, and ALB; (e) PPP1R1B alone; (f) PPP1R1B with TTR; (g) TTR and CHI3L1 with one of: CTSD; and PPP1R1B; (h) PPP1R1B, TTR, CTSD with one of: CHI3L1; ALB; C4B; and complement C1q B chain (C1QB); (i) TTR, CHI3L1, and CTSD with one of: ALB; cytochrome C (CYCS); CNR1; and C1QB; (j) TTR, ALB, and CYCS with one of: C4B; and PPP1R1B; (k) TTR, CTSD, and CYCS with one of: ALB; and C1QB; (1) C4B, TTR, CTSD, and CNR1; or (m) C1QB, TTR, CTSD, and CNR1; wherein the biomarker panel is for monitoring progression and/or severity of HD, when comparing pre HD with manifest HD or early/mid HD.

Alternatively, the term “HD biomarker panel” is meant to include: (a) PDYN alone; (b) PDYN with PENK; (c) PDYN and IGHG1 with one of: PENK; and C1QB; (d) PENK, CNR1, and IGF2; (e) CNR1, C1QB, and IGHG1; (f) CNR1, PPP1R1B, APOE, and IGHG1; (g) CNR1, PPP1R1B, BDNF; APOE, and IGHG1; (h) CNR1, BDNF; APOE, IGF2, and indoleamine 2,3-dioxygenase 1 (IDO1); (i) CNR1, BDNF; C1QB, IGF2, and IDO1; (j) CNR1, PPP1R1B, BDNF; C1QB, and IGHG1; or (k) PDYN, CNR1, PPP1R1B, C1QB, and IGHG1; wherein the biomarker panel is for monitoring progression and/or severity of HD, when comparing early/mid HD with late HD.

A clinical practitioner may use tominersen, AMT-130, or ANX005 or similar therapy alone or in combination for the treatment of HD. The timing of that administration and continued use of the therapeutic may depend on monitoring of one or more of the HD biomarker panels described herein.

As used herein the term “mass spectrometry” (MS) as used herein is a powerful analytical tool for the identification, characterization and quantification of various biomolecules, including, small molecules, drug metabolites, peptides and proteins) in biological samples. Mass spectrometry assays are able to measure multiple analytes simultaneously, they have low volume requirements and reagent costs are minimal. Mass spectroscopy may also be coupled with other methodologies, chromatography and immunoaffinity. MS comes in many varieties, for example, gas chromatography coupled to mass spectrometry (GC-MS), liquid chromatography with mass spectrometry (LC-MS), electrospray ionization (ESI), matrix-assisted laser desorption/ionization (MALDI), matrix assisted laser desorption ionization-time of flight mass spectrometry (MALDI-TOF-MS), and liquid chromatography tandem mass spectrometry (LC-MS/MS).

As used herein the term “immunoassay” as used herein is an analytical method that measures the presence or concentration of a macromolecule (including proteins and peptides) or a small molecule in a solution through the use of antibodies and/or antigens. Immunoassays are able to measure multiple analytes simultaneously provided that the antibodies have specificity and that the detectable label provides a sufficient signal. However, there are even immunoassays that work in the absence of a label. Labels may be enzymes, radioactive isotopes, fluorogenic, electrochemiluminescent, or even DNA labeled. There are two common types of enzyme immunoassays (EIAs), enzyme-linked immunosorbent assays (ELISAs) and enzyme multiplied immunoassay technique (EMIT). Electrochemiluminescence (ECL) is used as a label, and can emit detectable light in response to electric current. Immunoassay detection methods labeling the components of the assay, include surface plasmon resonance (SPR) binding between an unlabeled antibody and antigens may be detected.

An “effective amount” of an active ingredient as described herein includes a therapeutically effective amount or a prophylactically effective amount. A “therapeutically effective amount” refers to an amount effective, at dosages and for periods of time as needed, to achieve the desired therapeutic result, such as reduced HD symptoms, delayed progression of HD, increased life span or increased life expectancy. A therapeutically effective amount of an active ingredient may vary according to factors such as the disease state, age, sex, and weight of the subject, and the ability of the active ingredient to elicit a desired response in the subject. Dosage regimens may be adjusted to provide the optimum therapeutic response. A therapeutically effective amount is also one in which any toxic or detrimental effects of the active ingredient are outweighed by the therapeutically beneficial effects. A “prophylactically effective amount” refers to an amount effective, at dosages and for periods of time necessary, to achieve the desired prophylactic result, such as reduced HD symptoms, delayed progression of HD, increased life span, increased life expectancy or prevention of the respiratory viral infection. Typically, a prophylactic dose is used in subjects prior to or at an earlier stage of disease, so that a prophylactically effective amount may be less than a therapeutically effective amount.

It is to be noted that dosage values may vary with the severity of the condition to be alleviated. For any particular subject, specific dosage regimens may be adjusted over time according to the individual need and the professional judgment of the person administering or supervising the administration of the compositions. Dosage ranges set forth herein are exemplary only and do not limit the dosage ranges that may be selected by medical practitioners. The amount of active ingredient(s) in the composition may vary according to factors such as the disease state as determined by one or more of the HD biomarker panels described herein or other measures of HD progression and/or severity, age, sex, and weight of the subject. Dosage regimens may be adjusted to provide the optimum therapeutic response. For example, a single bolus may be administered, several divided doses may be administered over time or the dose may be proportionally reduced or increased as indicated by the exigencies of the therapeutic situation. It may be advantageous to formulate parenteral compositions in dosage unit form for ease of administration and uniformity of dosage.

In general, active ingredients, as described herein, should be used without causing substantial toxicity. Toxicity of the active ingredients as described herein can be determined using standard techniques, for example, by testing in cell cultures or experimental animals and determining the therapeutic index, i.e., the ratio between the LD₅₀ (the dose lethal to 50% of the population) and the LD₁₀₀ (the dose lethal to 100% of the population). In some circumstances however, such as in severe disease conditions, it may be appropriate to administer substantial excesses of the active ingredients. Some active ingredients as described herein may be toxic at some concentrations. Titration studies may be used to determine toxic and non-toxic concentrations. Animal studies may be used to provide an indication if the active ingredients have any effects on other tissues.

An active ingredient, as described herein, may be administered to a subject. As used herein, a “subject” may be a human, non-human primate, rat, mouse, cow, horse, pig, sheep, goat, dog, cat, etc. The subject may be suspected of having or at risk for having diabetes, such as Huntington disease (HD).

Huntington's disease (HD) is characterized by clinical motor impairment (e.g., involuntary movements, poor coordination, parkinsonism), cognitive deficits, and psychiatric symptoms. An inhered expansion of the CAG triplet in the huntingtin gene causing a pathogenic gain-of-function of the mutant huntingtin (mHTT) protein has been identified. Biomarkers of HD may provide for stratification of patients; be informative for the initiation of preventive treatment in premanifest HD or to delay symptom onset; and may also be useful for the identification of peripheral pathogenic central nervous system cascades.

As used herein “pre-manifest HD” and “pre-HD” are used interchangeably to indicate a subject having an HD, but not showing symptoms thereof, in particular not displaying HD motor symptoms. While patients that have slight symptoms, but not yet manifesting unequivocal signs are often categorized as having perimanifest HD (periHD) or being in a prodromal stage of HD. Manifest HD or man-HD may be broken into early/mid and late HD also depending on the symptoms displayed.

Any terms not directly defined herein shall be understood to have the meanings commonly associated with them as understood within the art.

### MATERIALS AND METHODS

### Study Participants

A retrospective analysis of protein analytes was performed in CSF from sixteen manifest HD, eight premanifest HD and eight healthy control individuals recruited through The University of British Columbia's Centre for Huntington Disease. Premanifest HD (preHD) was defined as individuals with HTT CAG repeat expansions >36 and a unified Huntington's Disease Rating Scale (uHDRS) diagnostic confidence level (DCL)<3, whereas manifest HD (manHD) was defined as individuals with a HTT CAG repeat expansion >36 and a DCL of 4. HD mutation carriers refer to both preHD and manHD individuals. Healthy control individuals with no neurological abnormalities and HTT CAG repeat lengths <36 were selected to span the range of ages in HD mutation carriers.

Clinical outcomes including: total functional capacity (TFC), total motor score (TMS), verbal fluency (VF), symbol digit modality test (SMDT), and Stroop word reading (SWR) were scored by a trained neurologist using the UHDRS.¹⁵ CAG-age product (CAP) score or disease burden score was calculated using the formula: (CAG length−35.5)×age.⁵⁰ Predicted age of onset in premanifest HD mutation carriers was calculated using the formula: 21.54+EXP(9.556−0.146×CAG repeat length) and years to predicted onset was estimated by subtracting the individuals age.^{9, 51} Quantitative-Motor assessments (Q-Motor score) measures are capable of detecting motor signs in blinded cross-sectional and longitudinal analyses of manifest, prodromal, and premanifest HD cohorts up to two decades before clinical diagnosis ⁸⁵. The Q-motor score is based on five assessments (i.e. digimotography (finger tapping), dysdiadochomotography (pronation/supination hand tapping), manumotography (grip force), choreomotography (chorea analysis), and pedomotography (speed foot tapping)). Of particular significance are the motor scores associated with digimotography (finger-tapping particularly the inter-onset-interval (IOI)) and dysdiadochomotography (pronation/supination).

### CSF Collection

CSF samples from HD mutation carriers and control individuals were obtained by lumbar puncture, examined qualitatively by microscopy, and centrifuged to remove cells. The acellular supernatant was aliquoted and frozen at −80° C.

### Parallel-Reaction Monitoring Mass Spectrometry

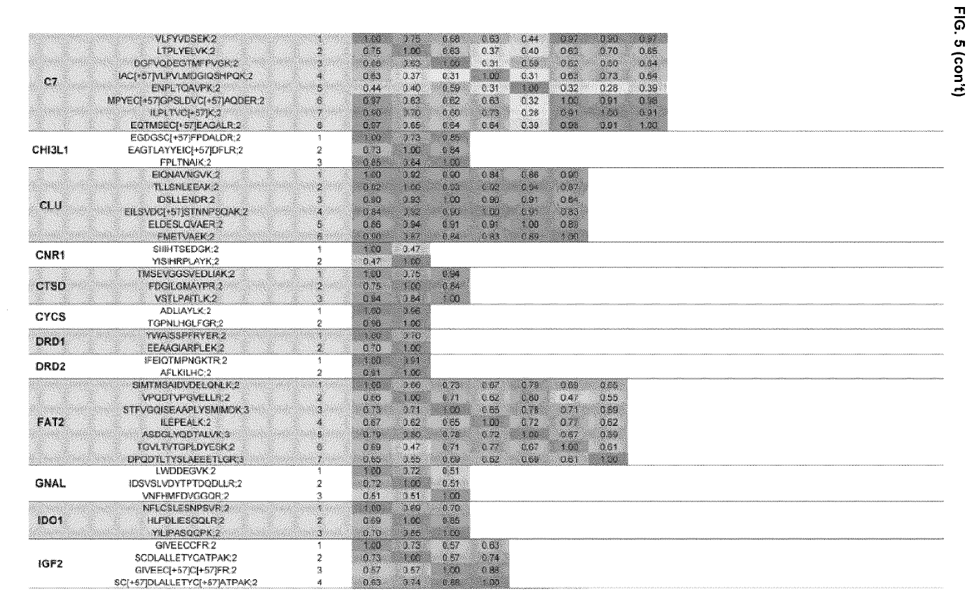

A panel of 26 proteins were measured in CSF by nanoLC-PRM-MS. For sample preparation, each CSF sample was reduced, alkylated, and trypsin digested as previously described ^{52,53} and cleaned using detergent removal spin columns (Thermo-Fisher™ Scientific, catalog #87777) as per the manufacturer's protocol. The samples were acidified with 1% formic acid (EMD Millipore) and loaded on a reversed-phase UltiMate™ 3000 RSLC-nano System with ProFlow Meter™ (Thermo-Fisher™) coupled with Orbitrap Eclipse™ Tribrid™ mass spectrometer (Thermo-Fisher™) for analysis with a nano-electrospray interface operated in positive ion mode. Prior to PRM analysis, 112 peptides corresponding to 2-15 peptides per protein (FIG. 5) were identified and validated using data-dependent acquisition (DDA) and split among four nanoLC-PRM-MS runs. The DDA and nanoLC-PRM-MS analysis involved injection and loading of approximately 0.1-0.2 μg of the peptide sample onto a 300 μm I.D.×0.5 mm 3 μm PepMaps™ C18 trap (Thermo-Fisher™) followed by separation on a 100 μm I.D.×10 cm 1.7 μm BEH130C18 nanoLC column (Waters™, Milford, MA). The eluted peptides were ionized by electrospray ionization for either DDA or nanoLC-PRM-MS analysis and the data for MS/MS was acquired in the Orbitrap™ on ions with mass-to-charge values between 375 and 1,800 at a resolution of 60,000 followed by higher-energy collisional dissociation (HCD) fragmentation and PRM scans. Raw data extraction and data analysis was performed using Skyline™ software version 3.7 (https://skyline.ms) and MatchRx™ software version 3.0 as previously described 53. The extracted peptide intensities (peak areas) were normalized against a median intensity value calculated from all peptide intensities in each run.

### Statistical Analysis

Statistical analyses were performed using GraphPad Prism 9™ (GraphPad™) and R statistical Software™, 54 using the Caret™,⁵⁵ and MixOmics™ ⁵⁶ packages for modelling. Alpha values of <0.05 were considered significant for all analyses.

Comparisons of demographic characteristics and clinical measures between groups were assessed by ANOVA and Fisher's least significant difference test. CAP scores were compared between preHD and manHD individuals using a two-tailed t-test. Differences in gender distributions between groups were assessed using Pearson's chi-squared (χ²) test.

Age, sex and CAG repeat length were considered potential confounding factors for comparisons of CSF protein levels between groups. The relationship of normalized CSF protein concentrations with age and sex were evaluated in control individuals using either Pearson's correlation or independent unpaired t-tests, respectively. The association of CSF protein levels with CAG repeat length in all HD mutation carriers was assessed using Pearson's correlation. Only age was found to be significantly associated with CSF protein levels and was included as a covariate for all subsequent analyses. Normalized CSF protein concentrations for all individuals were adjusted for age using linear regression.

Pre-specified analyses comparing age-adjusted CSF protein levels between controls and all HD mutation carriers were performed using general linear models (GLMs) bootstrapped with 1000 repetitions. P-values and the percentage of events in 1000 bootstrap repetitions that the variable was selected with P<0.05 are reported for each comparison. Odds ratio (OR) and 95% confidence intervals (CI) for statistically significant comparisons are presented.

Comparisons across disease stages were performed by ANCOVA including age as a covariate, and F statistics, degrees of freedom, and P-values for each comparison are reported. Post hoc tests between disease stages were performed using Tukey's test to correct for multiple comparisons and mean difference (MD) effect sizes, 95% CI and P-values for statistically significant comparisons are reported.

Associations of clinical measures of disease severity with CSF protein levels were assessed using Spearman's partial rank correlation including age as a covariate. Associations between each of the 26 CSF protein analytes were performed using Pearson's partial correlation including age as a covariate. Coefficient values (Spearman's p or Pearson's r) from +0.50 to +1 were considered strong correlations, +0.30 to +0.49 were considered moderate correlations and +0.10 to +0.29 were considered weak correlations. P-values <0.05 were used to define correlations significantly different than 0.

Sensitivity and specificity of each individual CSF protein for discriminating between disease groups/stages was assessed using receiver operating characteristic (ROC) curve analysis, and the corresponding area under the curve (AUC) values were computed as a measure of discriminatory performance or accuracy. CSF proteins with AUC=0.8-1 were considered as being classifiers with high discriminatory ability, values of 0.7-0.8 as having moderate discriminatory ability, and 0.6-0.7 as classifiers with weak discriminatory ability. AUC values, AUC 95% confidence intervals (CI) and P-values for each test are reported. AUC 95% CI were computed using the Wilson/Brown hybrid method and AUCs were compared as described by Delong and Delong.⁵⁷

Sparse partial least squares discriminant analysis (sPLS-DA) is a supervised machine learning method that examines the discriminative capacity of multi-dimensional data, while selecting features best able to classify samples. For each comparison, the sPLS-DA model was tuned to find the appropriate number of components and variables using 50×3-fold repeated cross-validation. Then, a final sPLS-DA model was fit using the optimal number of proteins for the respective optimal number of components, as determined during the tuning phase to avoid overfitting. This entire process was bootstrapped with 1000 repetitions to assess the variability and stability of the final models. ROC curves for the final sPLS-DA model were then generated and AUC values, AUC 95% CI and P-values are reported.

Multi-marker ROC curves were generated using the CombiROC analytical tool.⁵⁸ Data sets comprising age-adjusted values from up to 10 CSF proteins were uploaded into the web-based interface and analysis was performed without further processing of the data. Test-signal cutoffs as well as sensitivity and selectivity thresholds were adjusted for different group comparisons. ROC curves with combinations of up to 5 proteins were plotted and AUC values are reported.

### Examples

### Example 1: Demographics and Clinical Characteristics of Study Participants

Study participant demographics are summarized in TABLE 1. Our study included of eight healthy controls, eight preHD mutation carriers and sixteen manHD subjects. A significant age difference between groups was observed, with manHD patients being significant older than preHD individuals (52.12±11.94 vs. 37.18±9.08. P=0.011). Healthy controls were selected to span the age range of HD mutation carriers and no significant age differences were observed compared to either preHD (P=0.119) or manHD subjects (P=0.398). There were no significant differences in sex distributions between groups (χ²: 0.254, P=0.881) or CAG repeat lengths between preHD and manHD patients (43.64±1.51 vs. 44.50±2.78. P=0.196). Clinical scores for study participants are summarized in TABLE 1.

TABLE 1
Demographics and clinical characteristics of study participants.

| | Controls | PreHD | ManHD | | Controls | Controls | PreHD vs |
|---|---|---|---|---|---|---|---|
| | n = 8 | n = 8 | n = 16 | ANOVA | vs preHD | vs manHD | manHD |
| | mean ± SD | | | P-value^{a} | | | |
| Age | 47.63 ± 14.85 | 37.88 ± 9.08 | 52.13 ± 11.94 | 0.037 | 0.119 | 0.398 | 0.011 |
| Sex (M/F) | 4/4 | 5/3 | 9/7 | N/A | N/A | N/A | N/A |
| CAG | 17.88 ± 1.13 | 43.64 ± 1.51 | 44.50 ± 2.78 | <0.0001 | <0.0001 | <0.0001 | 0.196 |
| BMI | 27.06 ± 3.18 | 29.46 ± 7.81 | 26.20 ± 6.15 | 0.477 | 0.436 | 0.747 | 0.229 |
| DCL (n) | 0 (7), 1 (1) | 0 (2), 1 (5), 2 (1) | 4 (16) | N/A | N/A | N/A | N/A |
| CAP | N/A | 302.60 ± 65.65 | 492.80 ± 108.90 | N/A | N/A | N/A | 0.0002 |
| TFC | 13 ± 0 | 12.75 ± 0.46 | 5.25 ± 4.51 | <0.0001 | 0.879 | <0.0001 | <0.0001 |
| TMS | 0.38 ± 1.06 | 3.25 ± 2.96 | 57.81 ± 25.49 | <0.0001 | 0.757 | <0.0001 | <0.0001 |
| VF | 42.63 ± 9.13 | 43.63 ± 9.01 | 19.50 ± 17.40 | 0.0002 | 0.888 | 0.0007 | 0.0004 |
| SDMT | 51.25 ± 6.80 | 44.00 ± 9.35 | 18.56 ± 9.98 | <0.0001 | 0.124 | <0.0001 | <0.0001 |
| SWR | 96.38 ± 6.84 | 83.50 ± 15.48 | 53.81 ± 15.76 | <0.0001 | 0.077 | <0.0001 | <0.0001 |

^{a}P-values presented are not corrected for multiple comparisons
BMI = body mass index;
CAG = cytosine-adenine-guanine;
CAP = CAG-age product;
DCL = diagnostic confidence level;
manHD = manifest Huntington disease;
preHD = premanifest Huntington disease;
SD = standard deviation;
SDMT = symbol digit modality test;
SWR = Stroop word reading;
TFC = total functional capacity;
TMS = total motor score;
VF = verbal fluency

### Example 2: Comparison of CSF Protein Levels Across Disease Stages

We pre-specified 26 protein analytes to measure in CSF from controls and HD mutation carriers at different stages of disease, including protein analytes that have previously been investigated in HD CSF, as well as exploratory candidate proteins that, to our knowledge, have never been investigated in CSF from HD mutation carriers (TABLE 2).

TABLE 2
Protein analytes measured in CSF from HD mutation carriers and control individuals.

| | | Brain-enriched | Fold change^{a} | P-value (% | |
|---|---|---|---|---|---|
| CSF protein | Biological function(s) | expression | (HD/control) | selected)^{b} | |
| NEFL | Cytoskeleton/axonal transport | Yes | 1.97 | 0.031 | (60.2%) |
| GNAL^{c} | Signal transduction | Yes | 1.52 | 0.043 | (51.7%) |
| DRD1^{c} | Synaptic transmission/neuron growth | Yes | 1.42 | 0.062 | (44.5%) |
| IGF2^{c} | Carbohydrate metabolism/growth factor | No | 1.36 | 0.024 | (67.4%) |
| IGHG1 | Immune response | No | 1.28 | 0.015 | (77.6%) |
| CHI3L1 | Immune response | No | 1.25 | 0.371 | (2.5%) |
| C7^{c} | Complement system/immune response | No | 1.24 | 0.111 | (28.5%) |
| FAT2 | Cell adhesion/migration | Yes | 1.14 | 0.213 | (24.3%) |
| ALB | Transport protein | No | 1.14 | 0.068 | (42.1%) |
| PDE10A^{c} | Signal transduction | Yes | 1.07 | 0.611 | (4.8%) |
| CLU | Apoptosis/oxidative stress/immune response | No | 1.05 | 0.723 | (1.1%) |
| C4B | Complement system/immune response | No | 1.01 | 0.953 | (3.3%) |
| CYCS^{c} | Energy metabolism/apoptosis | No | 0.99 | 0.961 | (1.8%) |
| DRD2^{c} | Synaptic transmission/axonogenesis/neuron migration | Yes | 0.99 | 0.947 | (4.6%) |
| SIGMAR1^{c} | Lipid transport/G-protein signalling/apoptosis | No | 0.94 | 0.620 | (2.3%) |
| TTR | Signal transduction/transport protein | No | 0.90 | 0.314 | (11.4%) |
| C1QC | Complement system/immune response | No | 0.89 | 0.583 | (4.1%) |
| IDO1^{c} | Apoptosis/immune response | No | 0.89 | 0.156 | (22.8%) |
| CNR1^{c} | Apoptosis/synaptic transmission/immune response | Yes | 0.89 | 0.435 | (2.6%) |
| CTSD | Protein degradation/apoptosis/immune response | No | 0.87 | 0.044 | (52.3%) |
| C1QB^{c} | Complement system/immune response | No | 0.86 | 0.222 | (14.3%) |
| PPP1R1B^{c} | Signal transduction | Yes | 0.82 | 0.204 | (19.8%) |
| APOE^{c} | Lipid transport/synapse organization | No | 0.80 | 0.139 | (24.8%) |
| BDNF | Synapse assembly/axon guidance/neuronal health | Yes | 0.78 | 0.056 | (45.9%) |
| PDYN | Neuropeptide signalling | Yes | 0.76 | 0.018 | (65.6%) |
| PENK | Neuropeptide signalling | No | 0.63 | 0.011 | (84.9%) |

^{a}Fold changes represent the ratio of age-adjusted means between HD mutation carriers and controls. Colour scales were added to highlight increased (blue) or decreased (red) levels in HD mutation carriers
^{b}Represents the percentage of events in 1000 bootstrap repetitions that the variable was selected with a P-value <0.05.
^{c}Exploratory CSF markers not previously investigated in CSF from HD mutation carriers
HD = Huntington disease

We utilized a nanoLC-PRM-MS method to quantify unique peptides derived from each of the 26 CSF proteins with high sensitivity and specificity. For each protein, 2-15 unique peptides were measured in parallel. A complete list of peptide sequences measured by nanoLC-PRM-MS are presented in FIG. 5. We observed moderate to strong positive correlations between normalized unadjusted values for each peptide from all protein candidates assessed, suggesting a reliable measurement of these proteins in CSF (FIG. 5). Mean normalized peptide concentrations for each CSF protein were then adjusted to control for the effects of age, and residuals were used for all subsequent analyses.

We first compared age-adjusted values of CSF proteins in all HD mutation carriers (includes preHD and manHD individuals) and controls using bootstrapped GLMs (TABLE 2). We found that NEFL (OR=−2.785, 95% CI: −5.821 to −0.650, P=0.031), GNAL (OR=−3.134, 95% CI: −6.748 to −0.512, P=0.043), IGF2 (OR=−7.194, 95% CI: −14.816 to −1.798, P=0.024), and IGHG1 (OR=−8.026, 95% CI: −15.954 to −2.556, P=0.015) were significantly increased, whereas CTSD (OR=4.855, 95% CI: 0.821 to 10.688, P=0.044), PDYN (OR=3.912, 95% CI: 1.209 to 7.821, P=0.018), and PENK (OR=5.673, 95% CI: 2.301 to 11.464, P=0.011) were significantly decreased in CSF from HD mutation carriers. We also observed trends toward increased levels DRD1 (OR=−3.719, 95% CI: −8.216 to −0.194, P=0.062) and ALB (OR=−5.534, 95% CI: −12.505 to −0.258, P=0.068), and decreased levels of BDNF (OR=2.649, 95% CI: 0.13 to 5.716, P=0.056) in HD mutation carriers, but these did not reach statistical significance.

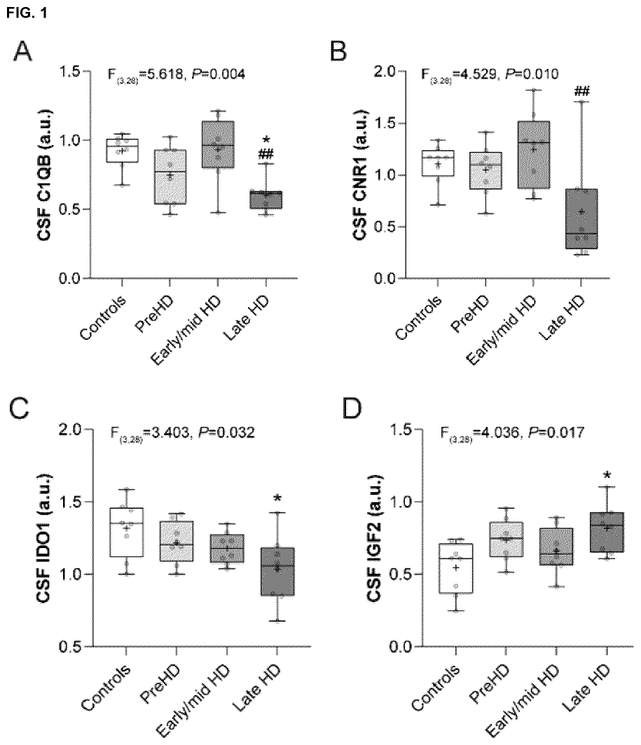

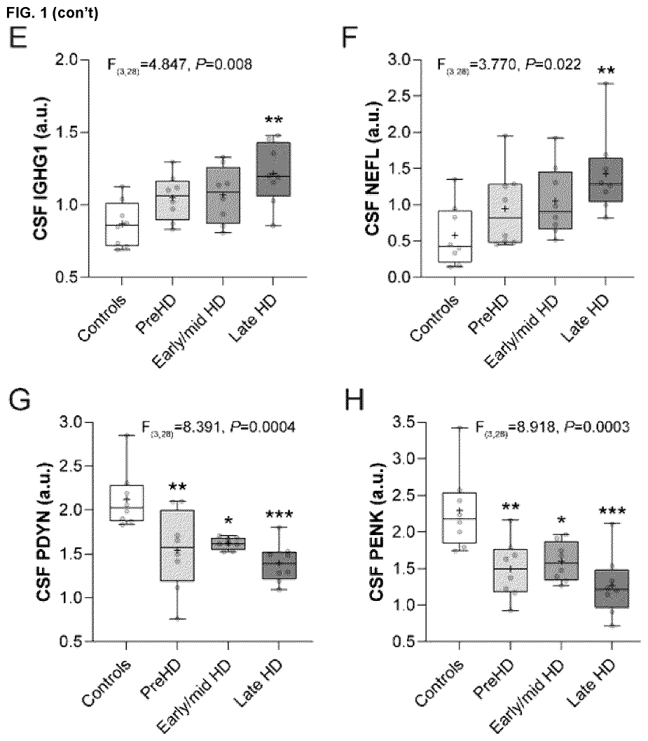

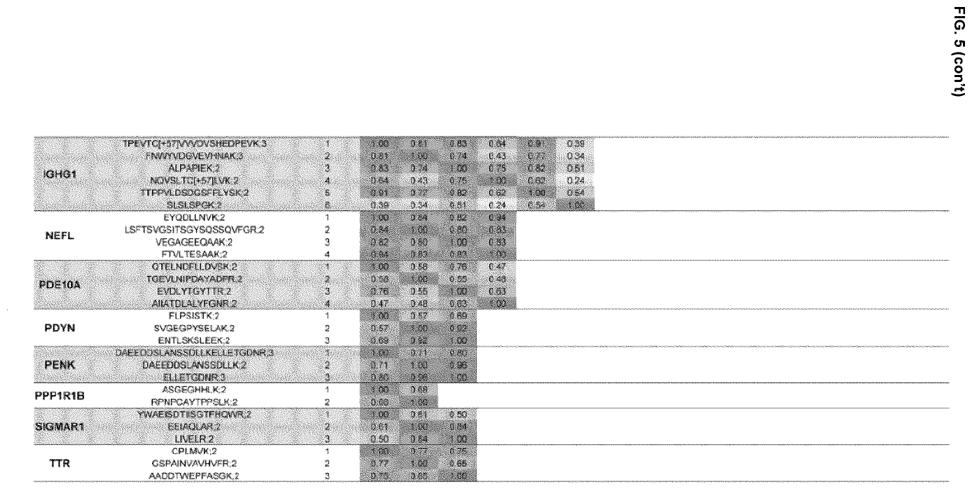

We next investigated whether CSF protein analyte levels were altered across disease stages. HD mutation carriers were divided based on DCL into preHD (DCL <3) and manHD groups (DCL=4), and the manHD group was further stratified based on TFC score into early/mid HD (TFC>5) and late HD (TFC<5) groups. A comparison of age-adjusted CSF protein levels was performed between controls, preHD, early/mid HD and late HD groups by ANCOVA followed by post hoc analysis using Tukey's test to correct for multiple comparisons (FIG. 6). We identified eight CSF proteins that were significantly altered across disease stages (FIG. 1).

Levels of C1QB were significantly decreased in late HD compared to controls (MD=0.322, 95% CI: 0.064 to 0.580, *P=0.010), and late HD compared to early/mid HD (FIG. 1A MD=0.331, 95% CI: 0.073 to 0.589, ##P=0.008). CNR1 levels were significantly reduced in late HD compared to early/mid HD (FIG. 1B MD=0.601, 95% CI: 0.133 to 1.070, ##P=0.008) and a strong trend towards a reduction in late HD compared controls was observed but did not reach post hoc significance (MD=0.461, 95% CI: −0.007 to 0.930, P=0.055).

IDO1 levels were significantly decreased (FIG. 1C MD=0.285, 95% CI: 0.036 to 0.533, *P=0.020), whereas IGF2 (FIG. 1D MD=−0.274, 95% CI: −0.497 to −0.050, *P=0.012), IGHG1 (FIG. 1E MD=−0.344, 95% CI: −0.592 to −0.097, ** P=0.004) and NEFL (FIG. 1F MD=−0.848, 95% CI: −1.368 to −0.327, ** P=0.002) were significantly increased in late HD compared to control individuals. Trends towards increased NEFL in early/mid HD compared to controls (MD=−0.473, 95% CI: −0.994 to 0.048, P=0.073) and late HD compared to preHD (MD=−0.483, 95% CI: −1.003 to 0.038, P=0.068) were observed but did not reach post hoc significance.

Levels of PDYN (FIG. 1G) and PENK (FIG. 1H) were significantly decreased in preHD (PDYN: MD=0.614, 95% CI: 0.176 to 1.052, ** P=0.004. PENK: MD=0.799, 95% CI: 0.229 to 1.369, ** P=0.004), early/mid HD (PDYN: MD=0.537, 95% CI: 0.099 to 0.975, *P=0.012, PENK: MD=0.698, 95% CI: 0.128 to 1.268, *P=0.012), and late HD compared to controls (PDYN: MD=0.761, 95% CI: 0.323 to 1.199, *** P=0.0003, PENK: MD=1.021, 95% CI: 0.451 to 1.591, *** P=0.0002).

PPP1R1B (also known as DARPP-32) levels were significantly changed across disease stages (FIG. 6 P=0.042) and showed a trend towards decreased levels in late HD compared to early/mid HD groups (MD=0.3682, 95% CI: −0.029 to 0.765, P=0.076). BDNF levels showed a strong trend towards a reduction in late HD compared to controls but this difference did not reach statistical significance (MD=0.433, 95% CI: −0.004 to 0.870, P=0.053).

### Example 3: Correlations of CSF Protein Levels with Clinical Measures of Disease Severity

Correlations of CSF protein levels with CAP score, an age-dependent measure of cumulative exposure to CAG expanded HTT, were performed on unadjusted values using Spearman's rank correlation in all HD mutation carriers (TABLE 3). C4B (ρ=0.44, P=0.031), NEFL (ρ=0.44, P=0.033), IDO1 (ρ=−0.45, P=0.029), and PENK (ρ=−0.41, P=0.048) showed significant correlations with CAP score.

TABLE 3

| | CAP | | TFC | | TMS | | VF | | SDMT | | SWR | |
|---|---|---|---|---|---|---|---|---|---|---|---|---|
| | ρ | P-value | ρ | P-value | ρ | P-value | ρ | P-value | ρ | P-value | ρ | P-value |
| ALB | −0.27 | 0.196 | 0.07 | 0.791 | 0.15 | 0.569 | 0.10 | 0.703 | −0.12 | 0.665 | 0.02 | 0.953 |
| APOE | −0.09 | 0.661 | 0.23 | 0.386 | 0.08 | 0.781 | 0.13 | 0.641 | 0.37 | 0.160 | −0.04 | 0.890 |
| BDNF | −0.08 | 0.698 | 0.45 | 0.083 | −0.25 | 0.356 | 0.32 | 0.222 | 0.48 | 0.064 | 0.21 | 0.422 |
| C1QB | 0.00 | 0.986 | 0.31 | 0.236 | −0.11 | 0.678 | 0.19 | 0.469 | 0.51 | 0.045 | 0.09 | 0.742 |
| C1QC | 0.11 | 0.623 | 0.00 | 0.998 | 0.30 | 0.252 | −0.13 | 0.637 | 0.14 | 0.594 | −0.27 | 0.303 |
| C4B | 0.44 | 0.031 | 0.34 | 0.196 | −0.37 | 0.164 | 0.10 | 0.699 | 0.31 | 0.236 | 0.28 | 0.287 |
| C7 | 0.22 | 0.295 | −0.09 | 0.749 | 0.29 | 0.279 | −0.27 | 0.308 | −0.04 | 0.881 | −0.36 | 0.175 |
| CHI3L1 | 0.40 | 0.054 | 0.30 | 0.252 | −0.03 | 0.910 | 0.03 | 0.912 | 0.38 | 0.142 | −0.10 | 0.720 |
| CLU | −0.01 | 0.965 | 0.12 | 0.645 | 0.07 | 0.807 | 0.07 | 0.802 | 0.31 | 0.236 | −0.08 | 0.767 |
| CNR1 | −0.24 | 0.264 | 0.58 | 0.021 | −0.38 | 0.142 | 0.52 | 0.041 | 0.56 | 0.026 | 0.28 | 0.292 |
| CTSD | −0.32 | 0.126 | 0.07 | 0.783 | −0.06 | 0.833 | 0.25 | 0.348 | 0.21 | 0.440 | 0.13 | 0.617 |
| CYCS | 0.24 | 0.258 | 0.08 | 0.757 | 0.21 | 0.437 | −0.09 | 0.737 | 0.20 | 0.447 | −0.18 | 0.492 |
| DRD1 | 0.10 | 0.658 | 0.05 | 0.857 | −0.18 | 0.495 | 0.12 | 0.649 | −0.10 | 0.713 | 0.11 | 0.670 |
| DRD2 | 0.15 | 0.473 | 0.27 | 0.312 | −0.42 | 0.106 | 0.18 | 0.494 | 0.24 | 0.359 | 0.25 | 0.349 |
| FAT2 | 0.14 | 0.509 | −0.05 | 0.852 | 0.22 | 0.411 | −0.14 | 0.600 | −0.01 | 0.984 | −0.21 | 0.442 |
| GNAL | 0.09 | 0.671 | −0.07 | 0.800 | −0.02 | 0.941 | 0.05 | 0.850 | −0.21 | 0.426 | 0.14 | 0.598 |
| IDO1 | −0.45 | 0.029 | 0.43 | 0.100 | −0.27 | 0.313 | 0.50 | 0.053 | 0.32 | 0.224 | 0.21 | 0.429 |
| IGF2 | 0.18 | 0.387 | −0.40 | 0.122 | 0.40 | 0.126 | −0.37 | 0.161 | −0.36 | 0.164 | −0.42 | 0.106 |
| IGHG1 | 0.07 | 0.759 | −0.28 | 0.285 | 0.24 | 0.365 | −0.15 | 0.589 | −0.33 | 0.205 | −0.15 | 0.570 |
| NEFL | 0.44 | 0.033 | −0.23 | 0.392 | 0.32 | 0.231 | −0.40 | 0.123 | −0.22 | 0.405 | −0.50 | 0.048 |
| PDE10A | 0.08 | 0.718 | 0.21 | 0.424 | −0.27 | 0.313 | 0.14 | 0.608 | 0.10 | 0.700 | 0.27 | 0.300 |
| PDYN | −0.26 | 0.212 | 0.53 | 0.035 | −0.36 | 0.171 | 0.55 | 0.031 | 0.57 | 0.023 | 0.43 | 0.100 |
| PENK | −0.41 | 0.048 | 0.35 | 0.182 | −0.05 | 0.858 | 0.31 | 0.238 | 0.42 | 0.111 | 0.14 | 0.605 |
| PPP1R1B | 0.12 | 0.563 | 0.54 | 0.034 | −0.34 | 0.200 | 0.41 | 0.113 | 0.53 | 0.038 | 0.23 | 0.393 |
| SIGMAR1 | −0.22 | 0.303 | 0.32 | 0.222 | −0.33 | 0.205 | 0.36 | 0.169 | 0.08 | 0.758 | 0.26 | 0.320 |
| TTR | −0.20 | 0.360 | 0.30 | 0.262 | −0.52 | 0.043 | 0.49 | 0.056 | 0.40 | 0.126 | 0.50 | 0.051 |

CAP = CAG-age product;
SDMT = symbol digit modality test;
SWR = Stroop word reading;
TFC = total functional capacity;
TMS = total motor score;
VF = verbal fluency

The relationship of CSF protein levels with clinical measures of disease severity in manHD individuals were evaluated using Spearman's partial rank correlation including age as a covariate (TABLE 3). CNR1 (ρ=0.58, P=0.021), PPP1R1B (ρ=0.54, P=0.034), and PDYN (ρ=0.53, P=0.035) were strongly correlated with TFC in manHD individuals, whereas BDNF (ρ=0.45, P=0.083), IDO1 (ρ=0.43, P=0.100) and IGF2 (ρ=−0.40, P=0.122) showed moderate correlations. TTR (ρ=−0.52, P=0.043) showed a strong significant negative correlation, and dopamine receptor D2 (DRD2; ρ=−0.42, P=0.106) and IGF2 (ρ=0.40, P=0.126) were moderately correlated with TMS in manHD subjects.

PDYN (ρ=0.55, P=0.031), CNR1 (ρ=0.52, P=0.041) and IDO1 (ρ=0.50, P=0.053) were strongly correlated with VF score, whereas TTR (ρ=0.49, P=0.056) and PPP1R1B (ρ=0.41, P=0.113) showed moderate positive correlations. PDYN (ρ=0.57, P=0.023), CNR1 (ρ=0.56, P=0.026), PPP1R1B (ρ=0.53, P=0.038), and C1QB (ρ=0.51, P=0.045) showed strong significant correlations, whereas BDNF (ρ=0.48, P=0.064), PENK (ρ=0.42, P=0.111) and TTR (ρ=0.40, P=0.126) showed moderate correlations with SDMT in manHD individuals. Finally, NEFL (ρ=−0.50, P=0.048) and TTR (ρ=0.50, P=0.051) showed strong correlations with SWR score, whereas PDYN (ρ=0.43, P=0.0997) and IGF2 (ρ=−0.42, P=0.1063) were moderately correlated with this clinical measure in manHD individuals.

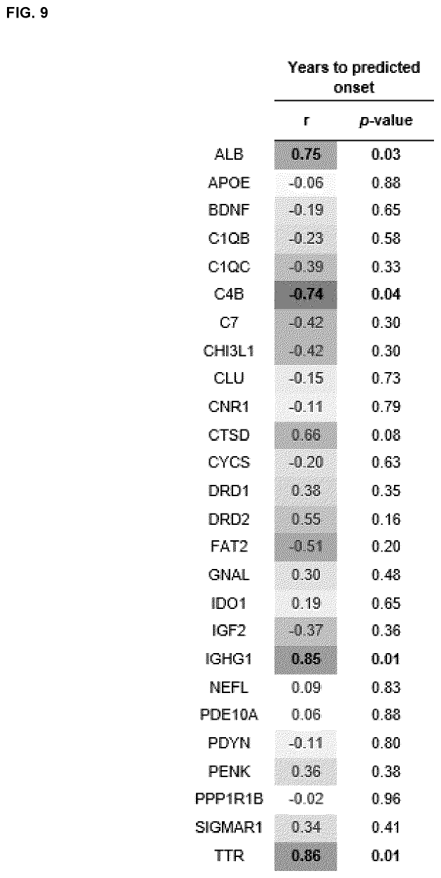

The relationship of unadjusted CSF protein levels and predicted age-of-onset of disease in premanifest HD individuals were evaluated using Pearson's correlation coefficients and p-values. Four proteins ALB (r=0.75, P=0.03), C4B (r=−0.74, P=0.04), IGHG1 (r=0.85, P=0.01) and TTR (r=0.86, P=0.01) showed significant correlation with age-of-onset (FIG. 9). These proteins were included in some of the combinations that showed perfect discrimination between preHD and early/mid HD.

### Example 4: Correlations Between CSF Protein Analytes in HD Mutation Carriers

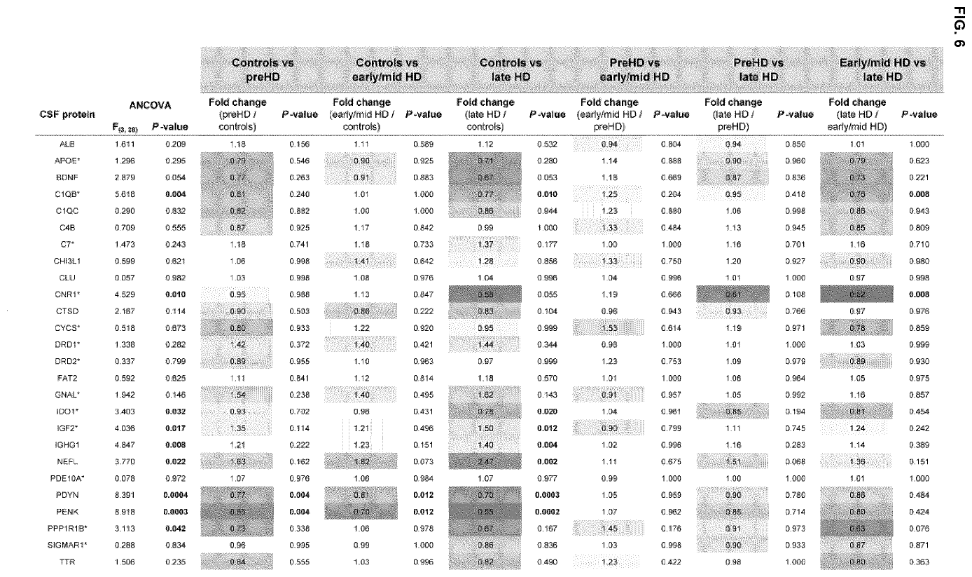

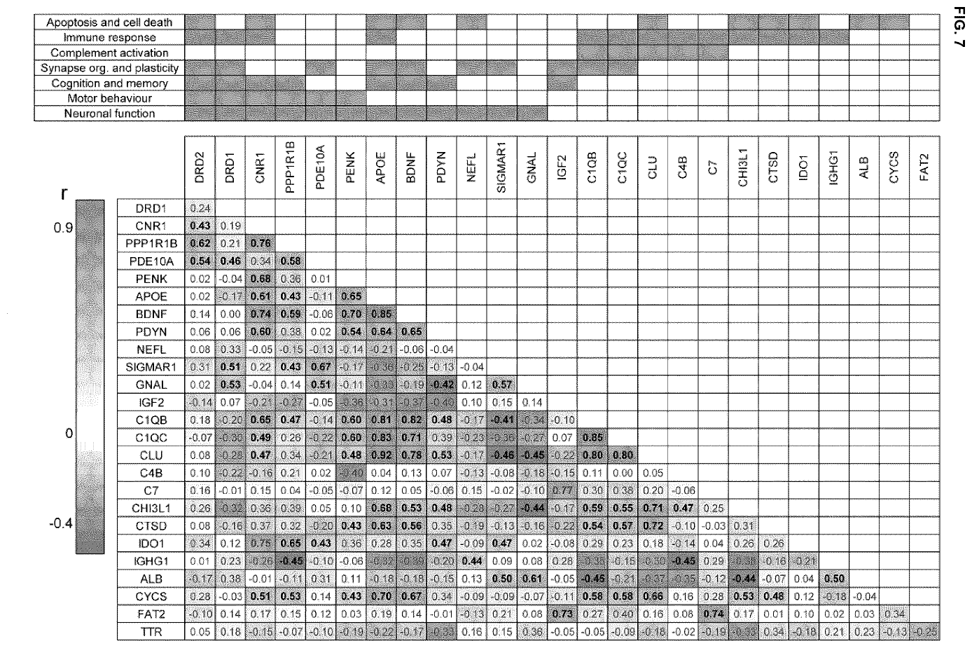

The relationship between individual CSF protein analytes in HD mutation carriers was assessed using Pearson's partial correlation (FIG. 7). Functional enrichment analysis was performed using all 26 CSF protein analytes to identify overlap in biological processes related to the pathophysiology of HD.⁵⁹ We observed moderate to strong correlations between CSF proteins involved in neuronal function, motor behaviour, cognition and memory, synapse organization and plasticity, apoptosis/cell death, as well as immune and complement pathway activation.

### Example 5: Discriminatory Potential of CSF Proteins for Distinguishing Subjects Based on HD Mutation Status and Disease Severity

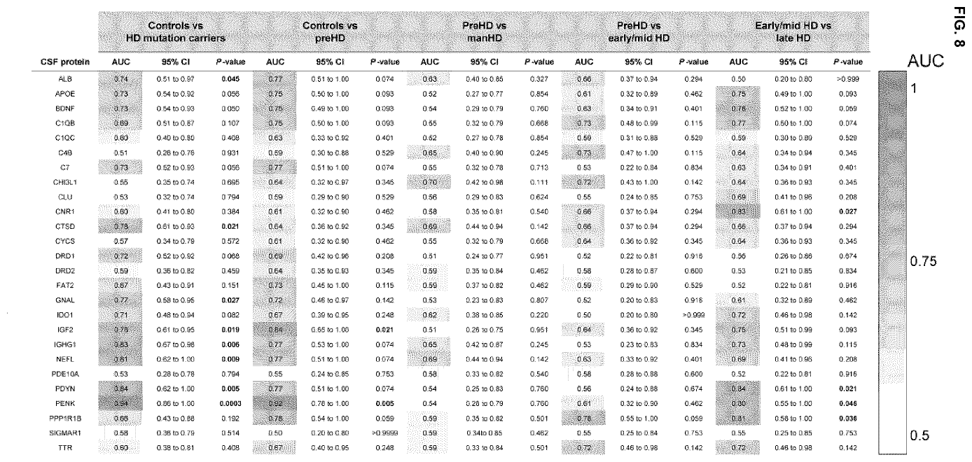

We next used ROC curve analysis to evaluate the sensitivity (% of individuals with the target condition that the test correctly identifies as positive) and specificity (% of individuals without the target condition that the test correctly identifies as negative) of each CSF protein for discriminating between either HD mutation carriers and controls, preHD and controls, or manHD and preHD. For each test, AUC values were computed as a measure of discriminatory performance for distinguishing individuals based on HD mutation status and disease severity (FIG. 8).

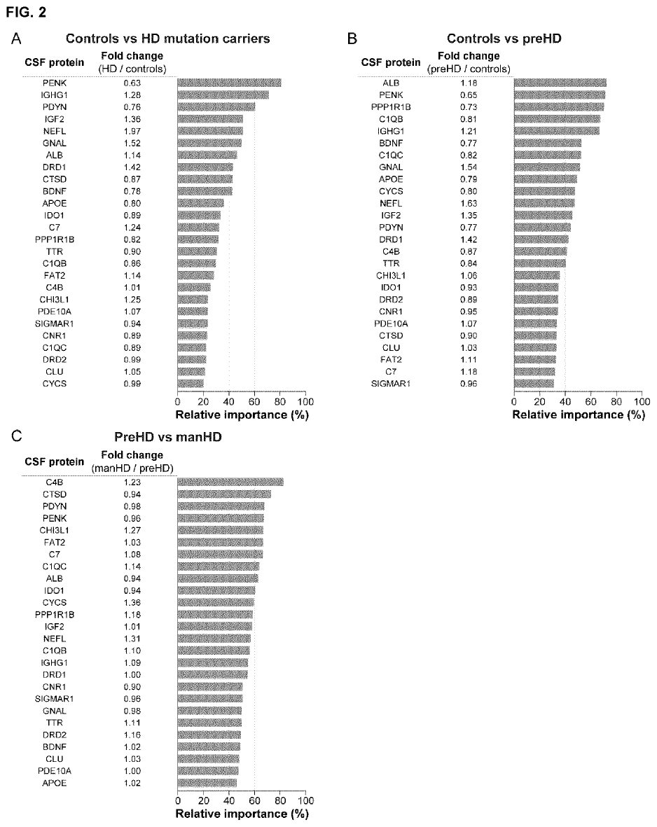

PENK showed the strongest discriminatory ability of any CSF protein for distinguishing between HD mutations carriers and controls, (FIG. 2A AUC=0.94, 95% CI: 0.86-1.00, P=0.0003), accurately classifying 79.2% of HD mutation carriers and 100% of control individuals. PENK also showed the highest discriminatory accuracy for distinguishing preHD from controls (FIG. 2B AUC=0.92, 95% CI: 0.78-1.00, P=0.005), correctly classifying 75% of preHD and 100% of control individuals. CHI3L1 showed only moderate discriminatory power for distinguishing between manHD and preHD individuals (FIG. 2C AUC=0.70, 95% CI: 0.42-0.98, P=0.111), accurately classifying 93.8% of manHD but only 62.5% of preHD individuals.

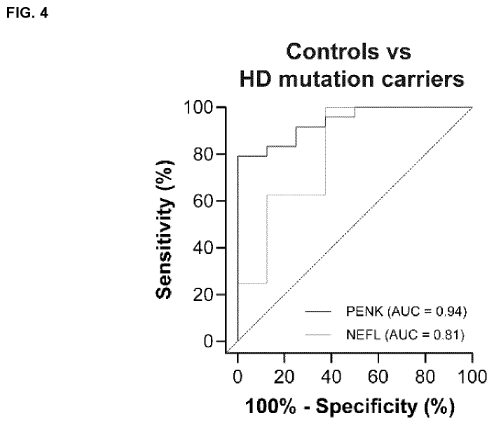

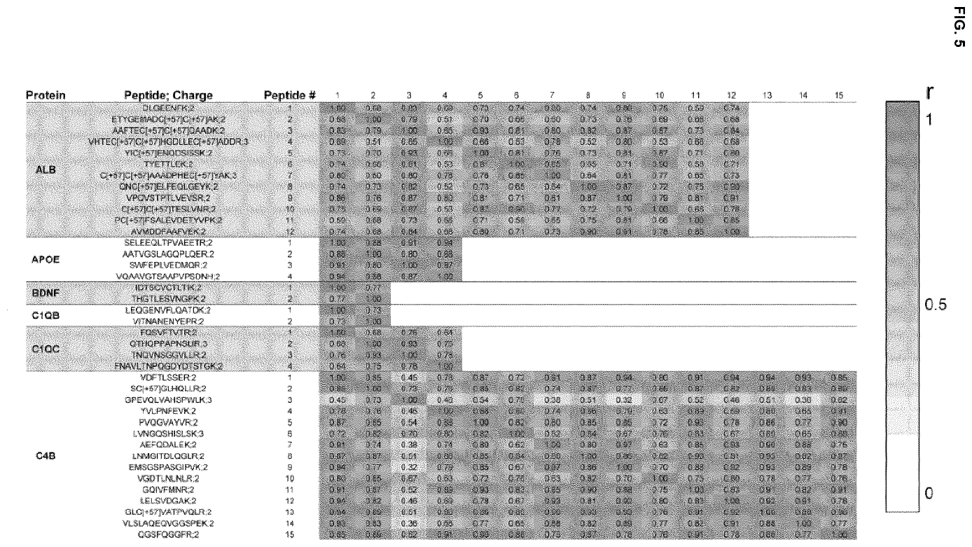

CSF NEFL was previously shown to have high accuracy for distinguishing between HD mutation carriers and controls as well as manHD and preHD groups.²³ We observed that NEFL showed strong discriminatory ability for distinguishing between HD mutation carriers and controls (FIG. 8. AUC=0.81, 95% CI: 0.62-1.00, P=0.009), but relatively weak discriminatory ability for distinguishing manHD from preHD in our cohort (FIG. 2C. AUC=0.69, 95% CI: 0.44-0.94, P=0.142). By comparison, PENK showed superior discriminatory ability to NEFL for distinguishing between HD mutation carriers and controls, but this did not reach statistical significance (FIG. 4. P=0.121).

We next performed sPLS-DA to evaluate the discriminatory potential of combining all 26 CSF proteins for distinguishing between between HD mutation carriers and controls, preHD and controls, or manHD and preHD. The relative discriminatory importance of individual CSF proteins to each sPLS-DA model are presented in FIG. 2.

A two-dimensional score plot generated using sPLS-DA segregated clusters, corresponding to HD mutation carriers and control individuals, along component 1 and 2 axes with minimal overlap (not shown). The model identified PENK (81%), IGHG1 (71.3%), PDYN (60.7%), IGF2 (51.3%) and NEFL (51.1%) as being the 5 most discriminant CSF proteins for distinguishing HD mutation carriers from controls based on the frequency of instances the protein was selected after bootstrapping (FIG. 2A). The ROC curve generated from the sPLS-DA model showed high discriminatory ability for distinguishing between HD mutation carriers and controls (AUC=0.90, 95% CI: 0.79-1.00, P=0.0006), similar to what was observed with PENK alone (FIG. 2A. AUC=0.94).

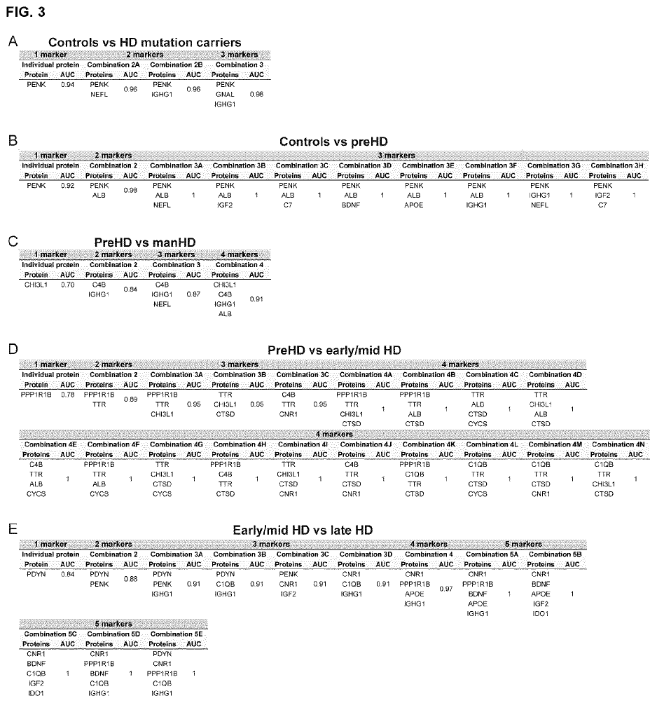

Dimensionality reduction using sPLS-DA segregated individuals from preHD and control groups with minimal overlap (not shown). The bootstrapped model identified ALB (72.3%), PENK (71.4%), PPP1R1B (70.1%), C1QB (67.3%) and IGHG1 (66.7%) as the 5 CSF proteins with the highest relative discriminatory importance (FIG. 2B). The ROC curve generated from the sPLS-DA model incorporating all 26 CSF proteins showed high discriminatory performance for distinguishing between preHD from control individuals (AUC=0.88, 95% CI: 0.69-1.00, P=0.010), similar to PENK alone (FIG. 3B. AUC=0.92).

The sPLS-DA model for discriminating manHD from preHD also showed strong segregation of groups on the two-dimensional score plot (not shown), and identified C4B (82.7%), CTSD (73%), PDYN (67.9%), PENK (67.3%) and CHI3L1 (66.9%) as having the highest relative discriminatory value (FIG. 2C). The ROC curve showed strong discriminatory performance for classifying manHD and preHD groups (AUC=0.95, 95% CI: 0.87-1.00, P=0.003), superior to CHI3L1 alone (FIG. 3C. AUC=0.70). These findings suggest that the combination of multiple CSF proteins can improve the discriminatory ability for distinguishing between manHD and preHD individuals.

### Example 6: Exploratory Multi-Marker CSF Protein Panels Improve Discrimination of Subjects Based on HD Mutation Status and Disease Severity

We next performed a combinatorial ROC curve analysis using the combiROC analytical tool⁵⁸ to identify marker combinations, comprising the fewest number of CSF proteins (up to 5), that could provide the highest discriminatory ability for distinguishing individuals based on HD mutation status and disease severity. All multi-marker combinations with the highest discriminatory accuracy are presented in FIG. 3.

The combination of PENK, IGHG1, and GNAL was able to accurately classify 88% of HD mutation carriers and 100% of control individuals, and improved discriminatory performance (FIG. 3A, AUC=0.98) beyond what was observed for any individual protein (FIG. 2A PENK AUC=0.94) or the combination of all 26 CSF proteins by sPLS-DA (AUC=0.90).

We identified eight unique combinations of 3 CSF proteins that showed perfect classification of preHD and controls, including combination 3A: PENK, ALB and NEFL (FIG. 3B AUC=1). These 3 marker panels showed superior discriminatory performance compared to PENK alone (FIG. 3B AUC=0.92) and the combination of all proteins (AUC=0.88)

The combination of CHI3L1, C4B, IGHG1, and ALB correctly classified 88% of preHD and 88% manHD individuals and showed discriminatory power (FIG. 3C AUC=0.91) similar to that observed using all 26 CSF proteins (AUC=0.95).

Finally, we wanted to define multi-marker CSF protein panels that could discriminate between individuals based on stratified disease stages. PPP1R1B showed the highest individual discriminatory accuracy for distinguishing between early/mid HD and preHD (FIG. 8 AUC=0.78, 95% CI: 0.55-1.00, P=0.059). Notably, fourteen unique combinations of 4 CSF proteins, including combination 4A: PPP1R1B, TTR, CHI3L1 and CTSD, showed perfect classification of preHD and early/mid HD individuals (FIG. 3D AUC=1).

PDYN showed the highest individual discriminatory ability for distinguishing late HD from early/mid HD individuals (FIG. 8. AUC=0.84, 95% CI: 0.61-1.00, P=0.021), whereas we identified five unique combinations of 5 CSF proteins that perfectly classified individuals with late HD and early/mid HD, including combination 5A: CNR1, PPP1R1B, BDNF, APOE and IGHG1 (FIG. 3E AUC=1).

Using nanoLC-PRM-MS, we quantified levels of 26 proteins in CSF from HD mutation carriers and healthy control individuals. Our primary objective was to replicate previously reported changes in CSF protein markers and to investigate whether novel candidate CSF proteins were altered in HD. Consistent with previous reports, we observed that NEFL,^{23,24,26-30} PENK,⁴⁰ PDYN,⁴¹ and CTSD³⁷ were significantly altered in the CSF of HD mutation carriers compared to controls after adjustment for age.

Multiple studies have reproducibly shown increased levels of blood and CSF NEFL in HD.^{23,24,26-30} Elevated levels of NEFL in biofluids have also been reported in other neurological diseases (reviewed in ³³) highlighting its utility as a biomarker of neuronal injury, but one that is not specific to HD. NEFL is currently being used in HD clinical trials as an exploratory biomarker of disease progression and to assess therapeutic efficacy.

We found NEFL levels to be significantly increased in the CSF of late HD subjects compared to control individuals (P=0.002), and trends towards elevated NEFL in early/mid HD compared to controls (P=0.073) and late HD compared to preHD (P=0.068). We did not however observe a significant increase of CSF NEFL in manHD compared to preHD, as reported previously using immunoassays to measure NEFL.^{23,26,29} We did however observe significant correlations of CSF NEFL with CAP (ρ=0.44) and SWR (ρ=−0.50) as reported previously.^{23,29} Our findings support the continued use of NEFL as an exploratory biomarker for monitoring disease severity and therapeutic response in clinical trials for HD.

PENK and PDYN are highly expressed in distinct striatal MSN populations² and are downregulated in the caudate of post mortem HD brains.⁴³ Both PENK and PDYN precursor proteins are cleaved to generate secreted peptides that modulate neurotransmission and regulate various neural functions in the brain. PENK levels in CSF were reported to be decreased in manHD compared to preHD and healthy controls using LC-MS.⁴⁰ We measured a significant reduction of PENK in preHD (P=0.004), early/mid HD (P=0.012) and late HD (P=0.0002) compared to controls and observed moderate correlations with CAP score (ρ=−0.48) and SDMT (ρ=0.42).

Reduced CSF PDYN was recently reported in manHD patients compared to controls using targeted LC-MS.⁴¹ This study found that levels of PDYN were not decreased in other neurodegenerative diseases, including: Alzheimer's disease, Parkinson's disease, and amyotrophic lateral sclerosis, suggesting that changes of CSF PDYN may be unique to HD.⁴¹ We found PDYN to be significantly reduced in preHD (P=0.004), early/mid HD (P=0.012) and late HD (P=0.0003) compared to controls. PDYN also showed strong associations with TFC (ρ=0.53), VF (ρ=0.55) and SDMT (ρ=0.57) in manHD individuals. We postulate that reduced CSF PENK and PDYN in preHD individuals may reflect early functional disturbances in the health of MSNs prior to disease-onset and differential loss of specific MSN sub-populations at more advanced stages of HD.

CTSD is a lysosomal protease expressed in the brain that has been shown to promote degradation of mHTT in primary neurons.⁶⁰ Levels of CTSD in the CSF were reported in one study to be significantly decreased in HD mutation carriers by MS³⁷ and in another to be unchanged between manHD, preHD and controls using PRM-MS.⁶¹ Consistent with these reports, we found CTSD to be significantly reduced in the CSF of HD mutation carriers compared to controls (P=0.044), but not significantly changed across disease stages.

In contrast to previous reports, we did not detect significant changes in C1QC,³⁷ C4B,³⁷ CHI3L1,^{29,37,38} CLU,^{39,40} FAT2,⁴⁰ or TTR^{37,40,42} protein levels in the CSF of HD mutation carriers compared to controls. These discordant findings could be due to differences in patient demographics and clinical characteristics, methodology used for detection of protein analytes in CSF, and/or the specific peptides that were selected for analysis by PRM-MS in our study.

BDNF is a growth factor required for the survival of various neuronal populations in the CNS and is downregulated in the caudate and putamen of HD patients compared to age-matched controls.⁶² Levels of BDNF in the CSF were previously reported to be unchanged across HD stages using an immunoassay.⁶³ We observed a strong trend towards a reduction of BDNF in late HD compared to controls (P=0.053) and moderate correlations with TFC (ρ=0.45) and SDMT (ρ=0.48). These findings suggest that reduced CSF BDNF may reflect depletion of BDNF production/release⁶⁴ or even the loss of cortical neurons at advanced stages of HD.⁵ Additional studies to investigate CSF BDNF as a potential biomarker for HD are warranted.

CSF to blood ALB^{65,66} and IgG quotients,⁶⁵ routinely used to measure blood-brain barrier (BBB)/blood-CSF barrier (BCSFB) dysfunction and intrathecal IgG production, were previously found to be unchanged in the CSF of HD mutation carriers compared to controls. Our data showed a strong trend towards increased ALB in HD mutation carriers compared to controls (P=0.068) and a significant increase of CSF IGHG1 (heavy chain constant domain of IgG) in the late HD compared to controls (P=0.004). The increased CSF albumin and IGHG1 could reflect neurovascular abnormalities and BBB/BCSFB dysfunction which have been reported in HD.⁶⁷⁻⁶⁹ Moreover, elevated CSF IGHG1 at advanced stages of HD may suggest increased local CNS IgG synthesis, a marker of CNS inflammation.

In addition to reproducing reported changes in previously investigated CSF biomarkers, we also identified novel candidate CSF proteins whose levels were altered in HD CSF. GNAL, which is highly expressed in the basal ganglia, plays an important role in MSN dopamine signaling.^{44,70} Reduced levels of GNAL have been reported in the caudate and putamen of HD patients.^{43,44} We found GNAL to be significantly elevated in the CSF of HD mutation carriers compared to controls (P=0.043), which could reflect an increased release from degenerating striatal MSNs in HD.

IGF2 is a regulator of neurogenesis, synaptic formation and spine maturation in the brain that plays a role in learning and memory functions.⁷¹⁻⁷³ Importantly, reduced IGF2 levels have been reported in striatum and plasma from HD patients.⁴⁹ We detected significantly elevated IFG2 levels in late HD compared to controls (P=0.012), and moderate correlations with TFC (ρ=−0.40), TMS (ρ=0.40), and SWR (ρ=−0.42) in manHD individuals. The unexpected increase of CSF IGF2 in HD mutation carriers is consistent with reports from Alzheimer's disease.^{74,75} We postulate that elevated CSF IGF2 may reflect increased release from IGF2 producing cells (eg. neural stem cells⁷³) or potentially a compensatory neuroprotective mechanism in the brain.

CNR1 is highly expressed in the basal ganglia where it modulates synaptic functions involved in motor behaviour.⁷⁶ Early downregulation of CNR1 has been reported in the striatum of HD patients.^{4,43} Levels of CNR1 were decreased in late HD compared to controls (P=0.055) and preHD (P=0.108), and were significantly reduced in CSF from late HD compared early/mid HD (P=0.008). Moreover, CSF CNR1 levels were strongly correlated with TFC (ρ=0.58), VF (ρ=0.52) and SDMT (ρ=0.56) in manHD. Reduced CSF CNR1 could be a marker that reflects the loss of CNR1-expressing neurons in the basal ganglia at advanced stages of HD.

C1Q (composed of A, B and C polypeptide chains), a component of the complement C1 recognition complex of the classical pathway, is released from CNS cells in response to inflammatory stimuli in neurodegenerative diseases.⁷⁷ In HD, upregulation of early complement activators and regulators from reactive microglia has been reported in the striatum of HD patients.⁷⁸ We found CSF C1QB to be modestly increased in early/mid compared to preHD and significantly reduced in late HD compared to early/mid HD (P=0.008) and controls (P=0.010). Surprisingly, we did not find C1QC to be significantly altered, although similar trends were observed. C1QB also showed a strong association with SDMT (ρ=0.51) in manHD individuals. These findings suggest early HD-associated complement activation in the brain, and potential dysregulation of this pathway at more advanced stages of disease.

IDO1, a rate-limiting enzyme in the kynurenine pathway, was reported to have increased expression and activity in the striatum of an HD mouse model.⁴⁸ IDO1 levels were significantly decreased in late HD compared to controls (P=0.020), and showed moderate to strong correlations with CAP score (ρ=−0.45), TFC (ρ=0.43) and VF (ρ=0.50). The reduction of IDO1 in the CSF could suggest dysregulation of the kynurenine pathway in the brain or may be a marker of cell loss in the striatum in late stage HD.

Together our data suggests that GNAL, IGF2, CNR1, C1QB, and IDO1 may represent promising CSF biomarker candidates that reflect distinct HD-associated pathophysiological alterations in the CNS.

A secondary objective of our study was to compare the discriminatory potential of individual CSF markers and combinations of CSF markers for distinguishing individuals based on HD mutation status and disease severity. We identified PENK and PDYN as being the most discriminant individual CSF proteins for distinguishing HD mutation carriers from controls. Notably, PENK (AUC=0.94) and PDYN (AUC=0.84) each showed superior discrimination of HD mutation carriers from controls compared to NEFL alone (AUC=0.81). Moreover, PENK (AUC=0.92) also showed the highest discriminatory power for distinguishing preHD from controls. No individual CSF protein showed high discriminatory accuracy for distinguishing between preHD and manHD individuals in our cohort, with only CHI3L1 (AUC=0.70) showing moderate discriminatory power.

sPLS-DA models incorporating all 26 CSF markers used to classify between either HD mutation carriers and controls (AUC=0.90) or preHD and controls (AUC=0.88) showed discriminatory performances similar to PENK alone (HD mutation carriers vs controls AUC=0.94, preHD vs controls AUC=0.92). However, a combination of all CSF markers improved discrimination of manHD from preHD (AUC=0.95) compared to CHI3L1 alone (AUC=0.70), highlighting the potential additive value of combining multiple CSF markers for distinguishing individuals based on severity of disease.

We also performed a combinatorial ROC curve analysis and defined exploratory multi-marker CSF panels with up to 5 proteins that, in all instances, showed superior discriminatory performance compared to individual proteins for distinguishing individuals based on HD mutation status and disease severity.

The combination of PENK, NEFL and ALB showed perfect discrimination between preHD and control individuals in our cohort suggesting that changes in these CSF proteins represent early events in disease pathogenesis, prior to overt symptomatic onset. Furthermore, all eight best 3 marker combinations included PENK, highlighting the importance of this marker for distinguishing between preHD and controls.

The panel consisting of CHI3L1, C4B, IGHG1, and ALB showed high discriminatory power (AUC=0.91) for distinguishing preHD from manHD individuals, with sensitivity and specificity superior to CHI3L1 alone (AUC=0.70) and similar to that observed with all 26 CSF markers by sPLS-DA (AUC=0.95). These data highlight the additive classification performance that is possible even when combining markers that individually have weak or moderate discriminatory ability.

Moreover, we identified fourteen unique 4 marker CSF protein panels that showed perfect discrimination of early/mid HD from preHD individuals, including the combination of C4B, TTR, ALB, and CYCS. Notably, ALB (r=0.75), C4B (r=−0.74), and TTR (r=0.86) were strongly correlated with predicted years to onset⁵¹ in preHD individuals. We postulate that this panel of CSF markers could be used in conjunction with CAG repeat length to improve accuracy of disease-onset predictions.

Finally, we identified multiple CSF marker panels, including the combination of CNR1, PPP1R1B, BDNF, APOE, and IGHG1, that showed perfect classification of late HD and early/mid HD individuals. This panel may reflect alterations in neuronal health, neurotrophic support, lipid metabolism, neuroinflammation, and BBB/BCSFB integrity that are associated with progression of HD.

Multi-marker CSF protein panels that can accurately discriminate between preHD and early/mid HD or manHD individuals could help define the optimal timing of therapeutic intervention for future clinical trials

Given the complex pathogenesis of HD and associated alterations of numerous biological pathways over the natural history of disease, it is likely that combinations of molecular biomarkers assessing multiple processes related to HD pathophysiology in parallel will be favoured for use in clinical trials to complement existing clinical and imaging biomarkers. Such panels could provide additional cell-type or pathway-specific resolution into HD-associated pathophysiological changes compared to a single biomarker, such as NEFL, which likely reflects general axonal damage/neuronal injury in the CNS.

MS-based methods are capable of sensitive detection of proteins in biofluids, comparable to other analytical assays, but may provide superior specificity through identification of multiple specific peptide sequences for any individual protein.^{79,80} Furthermore, targeted MS methods have high multiplexing capability (>100 peptides per assay) which is difficult to achieve with conventional assays (eg. immunoassays).⁸¹ Although MS-based assays may not be practical or cost-effective for routine clinical use, the exploratory multi-marker CSF protein panels identified in this study could be used to help guide the design of multiplex immunoassays that would be more amenable to clinical practice. The identification of multiple unique CSF protein combinations that are different in composition but that show equivalent discriminatory performance for classifying across disease stages provides flexibility for assay development and may help validation of such assays for clinical use.

We show evidence to suggest that combinations of CSF markers can outperform individual markers for classifying individuals based on HD mutation status and disease severity. Moreover, we define exploratory multi-marker CSF protein panels that we postulate may be useful for improving the accuracy of age-of-onset predictions for HD and complement clinical biomarkers for monitoring disease severity.

Various alternative embodiments and examples are described herein. These embodiments and examples are illustrative and should not be construed as limiting the scope of the invention.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Various alternative embodiments and examples are described herein. These embodiments and examples are illustrative and should not be construed as limiting the scope of the invention.

Abbreviations: AUC=area under the curve; BBB=blood-brain barrier; BCSFB=blood-CSF barrier; BMI=body mass index; CAG=cytosine-adenine-guanine; CAP=CAG-age product; CI=confidence interval; DCL=diagnostic confidence level; DDA=data-dependent acquisition; GLM=general linear model; HCD=higher-energy collisional dissociation; HD=Huntington disease; manHD=manifest Huntington disease; MD=mean difference; mHTT=mutant huntingtin; nanoLC-PRM-MS=nanoflow HPLC-coupled parallel-reaction monitoring mass spectrometry; OR=odds ratio; preHD=premanifest Huntington disease; ROC=receiver operating characteristic; SDMT=symbol digit modality test; sPLS-DA=sparse partial least square discriminant analysis; SWR=Stroop word reading; TFC=total functional capacity; TMS=total motor score; UHDRS=unified Huntington's disease rating scale; VF=verbal fluency

### REFERENCES

1. A novel gene containing a trinucleotide repeat that is expanded and unstable on Huntington's disease chromosomes. The Huntington's Disease Collaborative Research Group. Cell. Mar. 26 1993; 72 (6): 971-83. doi: 10.1016/0092-8674 (93) 90585-e
2. Deng Y P, Albin R L, Penney J B, Young A B, Anderson K D, Reiner A. Differential loss of striatal projection systems in Huntington's disease: a quantitative immunohistochemical study. J Chem Neuroanat. June 2004; 27 (3): 143-64. doi: 10.1016/j.jchemneu.2004.02.005
3. Reiner A, Albin R L, Anderson K D, D'Amato C J, Penney J B, Young A B. Differential loss of striatal projection neurons in Huntington disease. Proc Natl Acad Sci USA. August 1988; 85 (15): 5733-7. doi: 10.1073/pnas.85.15.5733
4. Glass M, Dragunow M, Faull R L. The pattern of neurodegeneration in Huntington's disease: a comparative study of cannabinoid, dopamine, adenosine and GABA (A) receptor alterations in the human basal ganglia in Huntington's disease. Neuroscience. 2000; 97 (3): 505-19. doi: 10.1016/s0306-4522 (00) 00008-7
5. Cudkowicz M, Kowall N W. Degeneration of pyramidal projection neurons in Huntington's disease cortex. Ann Neurol. February 1990; 27 (2): 200-4. doi: 10.1002/ana.410270217
6. Hedreen J C, Peyser C E, Folstein S E, Ross C A. Neuronal loss in layers V and VI of cerebral cortex in Huntington's disease. Neurosci Lett. Dec. 9 1991; 133 (2): 257-61. doi: 10.1016/0304-3940 (91) 90583-f
7. Heinsen H, Strik M, Bauer M, et al. Cortical and striatal neurone number in Huntington's disease. Acta Neuropathol. 1994; 88 (4): 320-33. doi: 10.1007/BF00310376
8. Ross C A, Aylward E H, Wild E J, et al. Huntington disease: natural history, biomarkers and prospects for therapeutics. Nat Rev Neurol. April 2014; 10 (4): 204-16. doi: 10.1038/nrneurol.2014.24
9. Langbehn D R, Brinkman R R, Falush D, Paulsen J S, Hayden M R, International Huntington's Disease Collaborative G. A new model for prediction of the age of onset and penetrance for Huntington's disease based on CAG length. Clin Genet. April 2004; 65 (4): 267-77. doi: 10.1111/j.1399-0004.2004.00241.x
10. Brinkman R R, Mezei M M, Theilmann J, Almqvist E, Hayden M R. The likelihood of being affected with Huntington disease by a particular age, for a specific CAG size. Am J Hum Genet. May 1997; 60 (5): 1202-10.
11. Genetic Modifiers of Huntington's Disease C. Identification of Genetic Factors that Modify Clinical Onset of Huntington's Disease. Cell. Jul. 30 2015; 162 (3): 516-26. doi: 10.1016/j.cell.2015.07.003
12. Genetic Modifiers of Huntington's Disease Consortium. Electronic address ghmhe, Genetic Modifiers of Huntington's Disease C. CAG Repeat Not Polyglutamine Length Determines Timing of Huntington's Disease Onset. Cell. Aug. 8 2019; 178 (4): 887-900 e14. doi: 10.1016/j.cell.2019.06.036
13. Wright GEB, Collins J A, Kay C, et al. Length of Uninterrupted CAG, Independent of Polyglutamine Size, Results in Increased Somatic Instability, Hastening Onset of Huntington Disease. Am J Hum Genet. Jun. 6 2019; 104 (6): 1116-1126. doi: 10.1016/j.ajhg.2019.04.007
14. Wexler N S, Lorimer J, Porter J, et al. Venezuelan kindreds reveal that genetic and environmental factors modulate Huntington's disease age of onset. Proc Natl Acad Sci USA. Mar. 9 2004; 101 (10): 3498-503. doi: 10.1073/pnas.0308679101
15. Unified Huntington's Disease Rating Scale: reliability and consistency. Huntington Study Group. Mov Disord. March 1996; 11 (2): 136-42. doi: 10.1002/mds.870110204
16. Schobel S A, Palermo G, Auinger P, et al. Motor, cognitive, and functional declines contribute to a single progressive factor in early H D. Neurology. Dec. 12 2017; 89 (24): 2495-2502. doi: 10.1212/WNL.0000000000004743
17. Tabrizi S J, Reilmann R, Roos R A, et al. Potential endpoints for clinical trials in premanifest and early Huntington's disease in the TRACK-H D study: analysis of 24 month observational data. Lancet Neurol. January 2012; 11 (1): 42-53. doi: 10.1016/S1474-4422 (11) 70263-0
18. Tabrizi S J, Scahill R I, Owen G, et al. Predictors of phenotypic progression and disease onset in premanifest and early-stage Huntington's disease in the TRACK-H D study: analysis of 36-month observational data. Lancet Neurol. July 2013; 12 (7): 637-49. doi: 10.1016/S1474-4422 (13) 70088-7
19. Byrne L M, Wild E J. Cerebrospinal Fluid Biomarkers for Huntington's Disease. J Huntingtons Dis. 2016; 5 (1): 1-13. doi: 10.3233/JHD-160196
20. Silajdzic E, Bjorkqvist M. A Critical Evaluation of Wet Biomarkers for Huntington's Disease: Current Status and Ways Forward. J Huntingtons Dis. 2018; 7 (2): 109-135. doi: 10.3233/JHD-170273
21. Southwell A L, Smith S E, Davis T R, et al. Ultrasensitive measurement of huntingtin protein in cerebrospinal fluid demonstrates increase with Huntington disease stage and decrease following brain huntingtin suppression. Sci Rep. Jul. 15 2015; 5:12166. doi: 10.1038/srep12166
22. Wild E J, Boggio R, Langbehn D, et al. Quantification of mutant huntingtin protein in cerebrospinal fluid from Huntington's disease patients. J Clin Invest. May 2015; 125 (5): 1979-86. doi: 10.1172/JCI80743
23. Byrne L M, Rodrigues F B, Johnson E B, et al. Evaluation of mutant huntingtin and neurofilament proteins as potential markers in Huntington's disease. Sci Transl Med. Sep. 12 2018; 10 (458) doi: 10.1126/scitranslmed.aat7108
24. Rodrigues F B, Byrne L M, Tortelli R, et al. Mutant huntingtin and neurofilament light have distinct longitudinal dynamics in Huntington's disease. Sci Transl Med. Dec. 16 2020; 12 (574) doi: 10.1126/scitranslmed.abc2888
25. Fodale V, Boggio R, Daldin M, et al. Validation of Ultrasensitive Mutant Huntingtin Detection in Human Cerebrospinal Fluid by Single Molecule Counting Immunoassay. J Huntingtons Dis. 2017; 6 (4): 349-361. doi: 10.3233/JHD-170269
26. Byrne L M, Rodrigues F B, Blennow K, et al. Neurofilament light protein in blood as a potential biomarker of neurodegeneration in Huntington's disease: a retrospective cohort analysis. Lancet Neurol. August 2017; 16 (8): 601-609. doi: 10.1016/S1474-4422 (17) 30124-2
27. Constantinescu R, Romer M, Oakes D, Rosengren L, Kieburtz K. Levels of the light subunit of neurofilament triplet protein in cerebrospinal fluid in Huntington's disease. Parkinsonism Relat Disord. March 2009; 15 (3): 245-8. doi: 10.1016/j.parkreldis.2008.05.012
28. Johnson E B, Byrne L M, Gregory S, et al. Neurofilament light protein in blood predicts regional atrophy in Huntington disease. Neurology. Feb. 20 2018; 90 (8): e717-e723. doi: 10.1212/WNL.0000000000005005
29. Vinther-Jensen T, Bornsen L, Budtz-Jorgensen E, et al. Selected CSF biomarkers indicate no evidence of early neuroinflammation in Huntington disease. Neurol Neuroimmunol Neuroinflamm. December 2016; 3 (6): e287. doi: 10.1212/NXI.0000000000000287
30. Parkin G M, Corey-Bloom J, Snell C, Castleton J, Thomas E A. Plasma neurofilament light in Huntington's disease: A marker for disease onset, but not symptom progression. Parkinsonism Relat Disord. June 2021; 87:32-38. doi: 10.1016/j.parkreldis.2021.04.017
31. Niemela V, Burman J, Blennow K, Zetterberg H, Larsson A, Sundblom J. Cerebrospinal fluid sCD27 levels indicate active T cell-mediated inflammation in premanifest Huntington's disease. PLOS One. 2018; 13 (2): e0193492. doi: 10.1371/journal.pone.0193492
32. Tabrizi S J, Leavitt B R, Landwehrmeyer G B, et al. Targeting Huntingtin Expression in Patients with Huntington's Disease. N Engl J Med. Jun. 13 2019; 380 (24): 2307-2316. doi: 10.1056/NEJMoa1900907
33. Yuan A, Nixon R A. Neurofilament Proteins as Biomarkers to Monitor Neurological Diseases and the Efficacy of Therapies. Front Neurosci. 2021; 15:689938. doi: 10.3389/fnins.2021.689938
34. Cifani P, Kentsis A. High Sensitivity Quantitative Proteomics Using Automated Multidimensional Nano-flow Chromatography and Accumulated Ion Monitoring on Quadrupole-Orbitrap-Linear Ion Trap Mass Spectrometer. Mol Cell Proteomics. November 2017; 16 (11): 2006-2016. doi: 10.1074/mcp.RA117.000023
35. Brzhozovskiy A, Kononikhin A, Bugrova A E, et al. The Parallel Reaction Monitoring-Parallel Accumulation-Serial Fragmentation (prm-PASEF) Approach for Multiplexed Absolute Quantitation of Proteins in Human Plasma. Anal Chem. Feb. 1 2022; 94 (4): 2016-2022. doi: 10.1021/acs.analchem.1c03782
36. Nguyen CDL, Malchow S, Reich S, et al. A sensitive and simple targeted proteomics approach to quantify transcription factor and membrane proteins of the unfolded protein response pathway in glioblastoma cells. Sci Rep. Jun. 20 2019; 9 (1): 8836. doi: 10.1038/s41598-019-45237-5
37. Fang Q, Strand A, Law W, et al. Brain-specific proteins decline in the cerebrospinal fluid of humans with Huntington disease. Mol Cell Proteomics. March 2009; 8 (3): 451-66. doi: 10.1074/mcp.M800231-MCP200
38. Rodrigues F B, Byrne L M, McColgan P, et al. Cerebrospinal Fluid Inflammatory Biomarkers Reflect Clinical Severity in Huntington's Disease. PLOS One. 2016; 11 (9): e0163479. doi: 10.1371/journal.pone.0163479
39. Dalrymple A, Wild E J, Joubert R, et al. Proteomic profiling of plasma in Huntington's disease reveals neuroinflammatory activation and biomarker candidates. J Proteome Res. July 2007; 6 (7): 2833-40. doi: 10.1021/pr0700753
40. Niemela V, Landtblom A M, Nyholm D, et al. Proenkephalin Decreases in Cerebrospinal Fluid with Symptom Progression of Huntington's Disease. Mov Disord. February 2021; 36 (2): 481-491. doi: 10.1002/mds.28391
41. Al Shweiki M R, Oeckl P, Pachollek A, et al. Cerebrospinal Fluid Levels of Prodynorphin-Derived Peptides are Decreased in Huntington's Disease. Mov Disord. February 2021; 36 (2): 492-497. doi: 10.1002/mds.28300
42. Vinther-Jensen T, Simonsen A H, Budtz-Jorgensen E, Hjermind L E, Nielsen J E. Ubiquitin: a potential cerebrospinal fluid progression marker in Huntington's disease. Eur J Neurol. October 2015; 22 (10): 1378-84. doi: 10.1111/ene.12750
43. Hodges A, Strand A D, Aragaki A K, et al. Regional and cellular gene expression changes in human Huntington's disease brain. Hum Mol Genet. Mar. 15 2006; 15 (6): 965-77. doi: 10.1093/hmg/ddl013
44. Corvol J C, Muriel M P, Valjent E, et al. Persistent increase in olfactory type G-protein alpha subunit levels may underlie D1 receptor functional hypersensitivity in Parkinson disease. J Neurosci. Aug. 4 2004; 24 (31): 7007-14. doi: 10.1523/JNEUROSCI.0676-04.2004
45. Ryskamp D, Wu J, Geva M, et al. The sigma-1 receptor mediates the beneficial effects of pridopidine in a mouse model of Huntington disease. Neurobiol Dis. January 2017; 97 (Pt A): 46-59. doi: 10.1016/j.nbd.2016.10.006
46. Luthi-Carter R, Strand A, Peters N L, et al. Decreased expression of striatal signaling genes in a mouse model of Huntington's disease. Hum Mol Genet. May 22 2000; 9 (9): 1259-71. doi: 10.1093/hmg/9.9.1259
47. Mazarei G, Neal S J, Becanovic K, Luthi-Carter R, Simpson E M, Leavitt B R. Expression analysis of novel striatal-enriched genes in Huntington disease. Hum Mol Genet. Feb. 15 2010; 19 (4): 609-22. doi: 10.1093/hmg/ddp527
48. Mazarei G, Budac D P, Lu G, et al. Age-dependent alterations of the kynurenine pathway in the YAC128 mouse model of Huntington disease. J Neurochem. December 2013; 127 (6): 852-67. doi: 10.1111/jnc.12350
49. Garcia-Huerta P, Troncoso-Escudero P, Wu D, et al. Insulin-like growth factor 2 (IGF2) protects against Huntington's disease through the extracellular disposal of protein aggregates. Acta Neuropathol. November 2020; 140 (5): 737-764. doi: 10.1007/s00401-020-02183-1
50. Penney J B, Jr., Vonsattel J P, MacDonald M E, Gusella J F, Myers R H. CAG repeat number governs the development rate of pathology in Huntington's disease. Ann Neurol. May 1997; 41 (5): 689-92. doi: 10.1002/ana.410410521
51. Langbehn D R, Hayden M R, Paulsen J S, and the P-HDIotHSG. CAG-repeat length and the age of onset in Huntington disease (H D): a review and validation study of statistical approaches. Am J Med Genet B Neuropsychiatr Genet. Mar. 5 2010; 153B (2): 397-408. doi: 10.1002/ajmg.b.30992
52. Haqqani A S, Caram-Salas N, Ding W, et al. Multiplexed evaluation of serum and CSF pharmacokinetics of brain-targeting single-domain antibodies using a NanoLC-SRM-ILIS method. Mol Pharm. May 6 2013; 10 (5): 1542-56. doi: 10.1021/mp3004995
53. Haqqani A S, Kelly J F, Stanimirovic D B. Quantitative protein profiling by mass spectrometry using label-free proteomics. Methods Mol Biol. 2008; 439:241-56. doi: 10.1007/978-1-59745-188-8_17
54. R: A language and environment for statistical computing. R Foundation for Statistical Computing, Vienna, Austria. https://www.R-project.org/. 2020.
55. Kuhn M. caret: Classification and Regression Training. R package version 6.0-90. https://CRAN.R-project.org/package=caret. 2021;
56. Rohart F, Gautier B, Singh A, Le Cao K A. mixOmics: An R package for ‘omics feature selection and multiple data integration. PLOS Comput Biol. November 2017; 13 (11): e1005752. doi: 10.1371/journal.pcbi.1005752
57. DeLong E R, DeLong D M, Clarke-Pearson D L. Comparing the areas under two or more correlated receiver operating characteristic curves: a nonparametric approach. Biometrics. September 1988; 44 (3): 837-45.
58. Mazzara S, Rossi R L, Grifantini R, Donizetti S, Abrignani S, Bombaci M. CombiROC: an interactive web tool for selecting accurate marker combinations of omics data. Sci Rep. Mar. 30 2017; 7:45477. doi: 10.1038/srep45477
59. Raudvere U, Kolberg L, Kuzmin I, et al. g: Profiler: a web server for functional enrichment analysis and conversions of gene lists (2019 update). Nucleic Acids Res. Jul. 2 2019; 47 (W1): W191-W198. doi: 10.1093/nar/gkz369
60. Liang Q, Ouyang X, Schneider L, Zhang J. Reduction of mutant huntingtin accumulation and toxicity by lysosomal cathepsins D and B in neurons. Mol Neurodegener. Jun. 1 2011; 6:37. doi: 10.1186/1750-1326-6-37
61. Lowe A J, Sjodin S, Rodrigues F B, et al. Cerebrospinal fluid endo-lysosomal proteins as potential biomarkers for Huntington's disease. PLOS One. 2020; 15 (8): e0233820. doi: 10.1371/journal.pone.0233820
62. Ferrer I, Goutan E, Marin C, Rey M J, Ribalta T. Brain-derived neurotrophic factor in Huntington disease. Brain Res. Jun. 2 2000; 866 (1-2): 257-61. doi: 10.1016/s0006-8993 (00) 02237-x
63. Ou Z A, Byrne L M, Rodrigues F B, et al. Brain-derived neurotrophic factor in cerebrospinal fluid and plasma is not a biomarker for Huntington's disease. Sci Rep. Feb. 10 2021; 11 (1): 3481. doi: 10.1038/s41598-021-83000-x
64. Zuccato C, Ciammola A, Rigamonti D, et al. Loss of huntingtin-mediated BDNF gene transcription in Huntington's disease. Science. Jul. 20 2001; 293 (5529): 493-8. doi: 10.1126/science.1059581
65. Jesse S, Brettschneider J, Sussmuth S D, et al. Summary of cerebrospinal fluid routine parameters in neurodegenerative diseases. J Neurol. June 2011; 258 (6): 1034-41. doi: 10.1007/s00415-010-5876-x
66. Huang Y C, Wu Y R, Tseng M Y, Chen Y C, Hsieh S Y, Chen C M. Increased prothrombin, apolipoprotein A-I V, and haptoglobin in the cerebrospinal fluid of patients with Huntington's disease. PLOS One. Jan. 31 2011; 6 (1): e15809. doi: 10.1371/journal.pone.0015809
67. Lin C Y, Hsu Y H, Lin M H, et al. Neurovascular abnormalities in humans and mice with Huntington's disease. Exp Neurol. December 2013; 250:20-30. doi: 10.1016/j.expneurol.2013.08.019
68. Drouin-Ouellet J, Sawiak S J, Cisbani G, et al. Cerebrovascular and blood-brain barrier impairments in Huntington's disease: Potential implications for its pathophysiology. Ann Neurol. August 2015; 78 (2): 160-77. doi: 10.1002/ana.24406
69. Di Pardo A, Amico E, Scalabri F, et al. Impairment of blood-brain barrier is an early event in R6/2 mouse model of Huntington Disease. Sci Rep. Jan. 24 2017; 7:41316. doi: 10.1038/srep41316
70. Neve K A, Seamans J K, Trantham-Davidson H. Dopamine receptor signaling. J Recept Signal Transduct Res. August 2004; 24 (3): 165-205. doi: 10.1081/rrs-200029981
71. Chen D Y, Stern S A, Garcia-Osta A, et al. A critical role for IGF-II in memory consolidation and enhancement. Nature. Jan. 27 2011; 469 (7331): 491-7. doi: 10.1038/nature09667
72. Schmeisser M J, Baumann B, Johannsen S, et al. IkappaB kinase/nuclear factor kappaB-dependent insulin-like growth factor 2 (Igf2) expression regulates synapse formation and spine maturation via Igf2 receptor signaling. J Neurosci. Apr. 18 2012; 32 (16): 5688-703. doi: 10.1523/JNEUROSCI.0111-12.2012
73. Bracko O, Singer T, Aigner S, et al. Gene expression profiling of neural stem cells and their neuronal progeny reveals IGF2 as a regulator of adult hippocampal neurogenesis. J Neurosci. Mar. 7 2012; 32 (10): 3376-87. doi: 10.1523/JNEUROSCI.4248-11.2012
74. Aberg D, Johansson P, Isgaard J, et al. Increased Cerebrospinal Fluid Level of Insulin-like Growth Factor-II in Male Patients with Alzheimer's Disease. J Alzheimers Dis. 2015; 48 (3): 637-46. doi: 10.3233/JAD-150351
75. Tham A, Nordberg A, Grissom F E, Carlsson-Skwirut C, Viitanen M, Sara V R. Insulin-like growth factors and insulin-like growth factor binding proteins in cerebrospinal fluid and serum of patients with dementia of the Alzheimer type. J Neural Transm Park Dis Dement Sect. 1993; 5 (3): 165-76. doi: 10.1007/BF02257671
76. Mievis S, Blum D, Ledent C. Worsening of Huntington disease phenotype in CB1 receptor knockout mice. Neurobiol Dis. June 2011; 42 (3): 524-9. doi: 10.1016/j.nbd.2011.03.006
77. Carpanini S M, Torvell M, Morgan B P. Therapeutic Inhibition of the Complement System in Diseases of the Central Nervous System. Front Immunol. 2019; 10:362. doi: 10.3389/fimmu.2019.00362
78. Singhrao S K, Neal J W, Morgan B P, Gasque P. Increased complement biosynthesis by microglia and complement activation on neurons in Huntington's disease. Exp Neurol. October 1999; 159 (2): 362-76. doi: 10.1006/exnr.1999.7170
79. Shi T, Fillmore T L, Sun X, et al. Antibody-free, targeted mass-spectrometric approach for quantification of proteins at low picogram per milliliter levels in human plasma/serum. Proc Natl Acad Sci USA. Sep. 18 2012; 109 (38): 15395-400. doi: 10.1073/pnas.1204366109
80. Yu Q, Paulo J A, Naverrete-Perea J, et al. Benchmarking the Orbitrap Tribrid Eclipse for Next Generation Multiplexed Proteomics. Anal Chem. May 5 2020; 92 (9): 6478-6485. doi: 10.1021/acs.analchem.9b05685
81. Gaither C, Popp R, Mohammed Y, Borchers C H. Determination of the concentration range for 267 proteins from 21 lots of commercial human plasma using highly multiplexed multiple reaction monitoring mass spectrometry. Analyst. May 18 2020; 145 (10): 3634-3644.
82. U.S. Pat. No. 10,174,321 uniQure I P B.V.
83. U S 2016/0090425 ANNEXON BIOSCIENCES
84. US2016/0159890 ANNEXON BIOSCIENCES
85. Reimann, R. and Schubert, R. Motor outcome measures in Huntington disease clinical trials. Handb Clin Neurol (2017) 144:209-225.

1. A Huntington Disease (HD) biomarker panel, the panel comprising:
(a) PENK alone;
(b) PENK with NEFL;
(c) PENK with IGHG1;
(d) PENK with GNAL and IGHG1;
(e) ALB alone;
(f) APOE alone;
(g) BDNF alone;
(h) C7 alone;
(i) CTSD alone;
(j) DRD1 alone;
(k) GNAL alone;
(l) IDO1 alone;
(m) IGF2 alone;
(n) IGHG1 alone;
(o) NEFL alone;
(p) PDYN alone; or
(q) combinations of any of the above;
wherein the biomarker panel indicative of biochemical changes in HD.

2. A HD biomarker panel, the panel comprising:
(a) ALB alone;
(b) C4B alone;
(c) IGHG1 alone;
(d) TTR alone;
(e) CNR1 alone;
(f) PDYN alone;
(g) PENK alone;
(h) PPP1R1B alone;
(i) APOE alone;
(j) BDNF alone;
(k) C1QB alone;
(l) C7 alone;
(m) FAT2 alone;
(n) GNAL alone;
(o) IGF2 alone;
(p) NEFL alone;
(q) PENK with ALB;
(r) PENK with ALB and one of NEFL, IGF2, C7, BDNF, APOE, and IGHG1;
(s) PENK with IGHG1 and NEFL; or
(t) PENK with IGF2 and C7;
wherein the biomarker panel is indicative of early biochemical changes in HD.

3. A HD biomarker panel, the panel comprising:
(a) CHI3L1 alone;
(b) C4B with IGHG1;
(c) C4B, IGHG1, and NEFL;
(d) CHI3L1 with C4B, IGHG1, and ALB;
(e) PPP1R1B alone;
(f) PPP1R1B with TTR;
(g) TTR and CHI3L1 with one of: CTSD; and PPP1R1B;
(h) C4B, TTR, and CNR1;
(i) PPP1R1B, TTR, CTSD with one of: CHI3L1; ALB; C4B; and C1QB;
(j) TTR, CHI3L1, and CTSD with one of: ALB; CYCS; CNR1; and C1QB;
(k) TTR, ALB, and CYCS with one of: C4B; and PPP1R1B;
(l) TTR, CTSD, and CYCS with one of: ALB; and C1QB;
(m) C4B, TTR, CTSD, and CNR1;
(n) C1QB, TTR, CTSD, and CNR1;
(o) C1QB alone;
(p) C4B alone; or
(q) TTR alone;
wherein the biomarker panel is for following the transition from pre-manifest HD to manifest HD.

4. A HD biomarker panel, the panel comprising:
(a) PDYN;
(b) PDYN with PENK;
(c) PDYN and IGHG1 with one of: PENK; and C1QB;
(d) PENK, CNR1, and IGF2;
(e) CNR1, C1QB, and IGHG1;
(f) CNR1, PPP1R1B, APOE, and IGHG1;
(g) CNR1, PPP1R1B, BDNF; APOE, and IGHG1;
(h) CNR1, BDNF; APOE, IGF2, and IDO1;
(i) CNR1, BDNF; C1QB, IGF2, and IDO1;
(j) CNR1, PPP1R1B, BDNF; C1QB, and IGHG1;
(k) PDYN, CNR1, PPP1R1B, C1QB, and IGHG1;
(l) APOE;
(m) BDNF;
(n) C1QB;
(o) CNR1;
(p) IDO1;
(q) IGF2;
(r) IGHG1;
(s) NEFL;
(t) PENK;
(u) PP1R1B; or
(v) TTR;
wherein the biomarker panel is for monitoring progression of HD or severity of HD or progression and severity of HD.

5. The HD biomarker panel of any one of claims 1-4, further comprising one or more clinical measures of disease severity selected from the following:
(i) a composite Unified Huntington's Disease Rating Scale (cUHDRS);
(ii) a Stroop word reading (SWR);
(iii) a symbol digit modality test (SDMT);
(iv) a total functional capacity (TFC);
(v) a total motor score (TMS); and
(vi) a Q-motor score.

6. The HD biomarker panel of any one of claims 1-4, wherein the HD biomarker panel is determined from a biological sample obtained from a human subject.

7. The HD biomarker panel of claim 6, wherein the biological sample is selected from the group consisting of: whole blood; blood plasma; blood serum; and cerebrospinal fluid (CSF).

8. The HD biomarker panel of any one of claims 1, 2, 5, 6 and 7, wherein the biomarker is selected from one or more of: NEFL; GNAL; DRD1; IGF2; IGHG1; CHI3L1; C7; FAT2; ALB; and C4B and wherein the one or more biomarkers show an increase in a cerebrospinal fluid (CSF) sample from a subject in comparison to a non-HD standard amount of protein as an indicator of HD severity and/or progression; or
wherein the biomarker is selected from one or more of: TTR; IDO1; CNR1; CTSD; C1QB; PPP1R1B; APOE; BDNF; PDYN; and PENK and wherein the one or more biomarkers show a decrease in a CSF sample from a subject in comparison to a non-HD standard amount of protein as an indicator of HD severity and/or progression.

9. The HD biomarker panel of any one of claims 1-8, wherein the HD biomarker panel is used to detect a target protein or a target peptide in a CSF sample using a mass spectrometry assay or an immunoassay.

10. The HD biomarker panel of claim 9, wherein the mass spectrometry assay is a nanoflow liquid chromatography-coupled parallel-reaction monitoring mass spectrometry (nanoLC-PRM-MS) and wherein the immunoassay is an enzyme linked immunoassay (ELISA).

11. A method of detecting the level of a biomarker panel in a subject suspected of having HD or known to have HD, the method comprising:
(a) measuring, in a biological sample obtained from the subject, a relative concentration of at least one biomarker in the biomarker panel, wherein the biomarker panel is set out in any one of claims 1-4; and
(b) monitoring biochemical changes in HD, monitoring progression of HD, or monitoring the severity of HD.

12. The method of claim 11, wherein said biological sample is selected from: CSF, whole blood; blood serum; and blood plasma.

13. The method of claim 12, wherein the biological sample is CSF.

14. The method of claim 11, 12, or 13, wherein the subject is a human.

15. The method of any one of claims 11-14, wherein the subject has been determined to have a CAG repeat expansion mutation in HTT.

16. The method of any one of claims 11-15, wherein the subject is assessed for biochemical changes in HD and wherein the biomarker panel is set out in claim 1 and where the biological sample has a:
(a) decreased PENK;
(b) decreased PENK and increased NEFL;
(c) decreased PENK and increased IGHG1;
(d) decreased PENK, increased GNAL, and increased IGHG1;
(e) increased ALB;
(f) decreased APOE;
(g) decreased BDNF;
(h) increased C7;
(i) decreased CTSD;
(j) increased DRD1;
(k) increased GNAL;
(l) decreased IDO1;
(m) increased IGF2;
(n) increased IGHG1;
(o) increased NEFL;
(p) decreased PDYN; or
(q) combinations of any of the above;
as compared to a control non-HD or to a non-HD standard at an approximately equivalent age.

17. The method of any one of claims 11-15, wherein the subject is assessed for early biochemical changes in HD and wherein the biomarker panel is set out in claim 2 and where the biological sample has a:
(a) increased ALB;
(b) increased C4B;
(c) increased IGHG1;
(d) decreased TTR;
(e) decreased CNR1;
(f) decreased PDYN;
(g) decreased PENK;
(h) decreased PPP1R1B;
(i) decreased APOE;
(j) decreased BDNF;
(k) decreased C1QB;
(l) increased C7;
(m) increased FAT2;
(n) increased GNAL;
(o) increased IGF2;
(p) increased NEFL;
(q) decreased PENK, increased ALB;
(r) decreased PENK, increased ALB and one of increased NEFL, increased IGF2, increased C7, decreased BDNF, decreased APOE, and increased IGHG1;
(s) decreased PENK, increased IGHG1 and increased NEFL; or
(t) decreased PENK, increased IGF2 and increased C7;
as compared to a control non-HD or to a non-HD standard at an approximately equivalent age.

18. The method of any one of claims 11-15, wherein the subject is assessed for following the transition from pre-manifest HD to manifest HD and wherein the biomarker panel is set out in claim 3 and where the biological sample has a:
(a) increased CHI3L1;
(b) increased C4B and increased IGHG1;
(c) increased C4B, increased IGHG1, and increased NEFL;
(d) increased CHI3L1, increased C4B, increased IGHG1, and decreased ALB;
(e) increased PPP1R1B;
(f) increased PPP1R1B and increased TTR;
(g) increased TTR, increased CHI3L1, and one of: decreased CTSD; and increased PPP1R1B;
(h) increased C4B, increased TTR, and decreased CNR1;
(i) increased PPP1R1B, increased TTR, decreased CTSD and one of: increased CHI3L1; decreased ALB; increased C4B; and increased C1QB;
(j) increased TTR, increased CHI3L1, decreased CTSD, and one of: decreased ALB; increased CYCS; decreased CNR1; and increased C1QB;
(k) increased TTR, decreased ALB, increased CYCS, and one of: increased C4B;
and increased PPP1R1B;
(l) increased TTR, decreased CTSD, increased CYCS, and one of: decreased ALB;
and increased C1QB;
(m) increased C4B, increased TTR, decreased CTSD, and decreased CNR1;
(n) increased C1QB, increased TTR, decreased CTSD, and decreased CNR1;
(o) increased C1QB;
(p) increased C4B; or
(q) increased TTR;
when manifest HD is compared to premanifest HD at an equivalent age.

19. The method of any one of claims 11-15, wherein the subject is assessed for monitoring progression of HD or severity of HD or progression and severity of HD and wherein the biomarker panel is set out in claim 4 and where the biological sample has a:
(a) decreased PDYN;
(b) decreased PDYN, and decreased PENK;
(c) decreased PDYN, and increased IGHG1 and one of: decreased PENK; and
decreased C1QB;
(d) decreased PENK, decreased CNR1, and increased IGF2;
(e) decreased CNR1, decreased C1QB, and increased IGHG1;
(f) decreased CNR1, decreased PPP1R1B, decreased APOE, and increased IGHG1;
(g) decreased CNR1, decreased PPP1R1B, decreased BDNF; decreased APOE, and increased IGHG1;
(h) decreased CNR1, decreased BDNF; decreased APOE, increased IGF2, and decreased IDO1;
(i) decreased CNR1, decreased BDNF; decreased C1QB, increased IGF2, and decreased ID01;
(j) decreased CNR1, decreased PPP1R1B, decreased BDNF; decreased C1QB, and increased IGHG1;
(k) decreased PDYN, decreased CNR1, decreased PPP1R1B, decreased C1QB, and increased IGHG1;
(l) decreased APOE;
(m) decreased BDNF;
(n) decreased C1QB;
(o) decreased CNR1;
(p) decreased ID01;
(q) increased IGF2;
(r) increased IGHG1;
(s) increased NEFL;
(t) decreased PENK;
(u) decreased PP1R1B; or
(v) decreased TTR;
when late HD is compared to early/mid HD at an equivalent age.

20. The method of any one of claims 11-19, wherein the subject known to have HD is further administered an HD treatment.

21. The method of any one of claims 11-19, wherein the subject known to have HD is further administered an HD treatment and the subject is further monitored for their response to the HD treatment based on biochemical changes as determined by testing of one or more of the biomarker panels of claims 1-4.

22. The method of claim 20 or 21, wherein the HD treatment is selected from one or more of: an antisense oligonucleotide, a siRNA, a miRNA, a small molecule, a CRISPR gene edit, wherein the HD treatment lowers levels of the mutant HTT protein in the CNS.

23. A method for monitoring response to treatment of HD and determining treatment efficacy in a subject, comprising the steps of:
(a) measuring levels of at least one biomarker in at least 2 longitudinal biological samples from the same subject and comparing the measured levels to an level of a matched biomarker determined in a clinically relevant population, wherein the at least one biomarker is from a first panel, comprising: NEFL; GNAL; DRD1; IGF2; IGHG1; CHI3L1; C7; FAT2; ALB; PDE10A; CLU; C4B; CYCS; DRD2; SIGMAR1; TTR; Q1QC; ID01; CNR1; CTSD; C1QB; PPP1R1B; APOE; BDNF; PDYN; and PENK, wherein the level of the one or more biomarkers in the biological sample is changed, and wherein at least one of the at least two biological samples is collected before the individual is treated for HD and at least one of the at least two biological samples is collected after the subject is treated for HD;
(b) calculating a score for the at least one biomarker in the biological samples, by summing: the number of biomarkers in the first panel exhibiting a change in level relative to the of the biomarker determined in a clinically relevant population, and/or the number of biomarkers in the second panel exhibiting a change in level relative to the of the biomarker determined in a clinically relevant population; and
(c) determining that said treatment(s) is effective if the score of the panel of biomarker(s) in the sample collected after treatment is lower than the score of at least one of the at least two biologicals samples collected before treatment.

24. The method of claim 23, wherein the at least one biomarker is from a panel, comprising: NEFL; GNAL; DRD1; IGF2; IGHG1; CHI3L1; C7; FAT2; ALB; PDE10A; CLU; C4B; CYCS; TTR; Q1QC; IDO1; CNR1; CTSD; C1QB; PPP1R1B; APOE; BDNF; PDYN; and PENK.
